(19) United States
(12) Patent Application Publication
(10) Pub. No.: US 2005/0288255 A1
(43) Pub. Date: Dec. 29, 2005

(54) Modulation of lysophosphatidylcholine and treatment of diet-induced conditions

(76) Inventors: David Hui, Cincinnati, OH (US); Dominique Charmot, Campbell, CA (US); Jerry M. Buysse, Los Altos, CA (US)

Correspondence Address: WILSON SONSINI GOODRICH & ROSATI, 650 PAGE MILL ROAD, PALO ALTO, CA, 94304-1050, US

(21) Appl. No.: 11/121,705

(22) Filed: May 3, 2005

Related U.S. Application Data
(60) Provisional application No. 60/568,066, filed on May 3, 2004.

(51) Int. Cl. A61K 31/685

(52) U.S. Cl. 514/78

(57) ABSTRACT

The present invention provides methods for the treatment of lysophosphatidylcholine-related conditions. In particular, the invention provides a method of treating an insulin-related condition, e.g., diabetes or diabetes type 2, and/or a weight-related-condition, e.g., unwanted weight gain or obesity, in an animal subject by reducing production, absorption and/or activity of lysophosphatidylcholine. Further, the beneficial effects of reducing lysophosphatidylcholine in terms of diabetes and weight gain are disclosed.

### RELATED APPLICATION

This application claims priority benefit of U.S. provisional patent application Ser. No. 60/568,066 entitled “Treatment of Diet-Induced Conditions” filed May 3, 2004 by Hui et al., which is incorporated herein by reference in its entirety for all purposes.

### BACKGROUND OF THE INVENTION

High fat diets and sedentary lifestyles of the industrialized world have lead to increasing incidence of diet-related health conditions. The digestion and absorption of lipids and phospholipids, for example, play important roles in conditions such as obesity and diabetes, although the mechanisms involved remain incompletely delineated.

Diabetes affects 18.2 million people in the Unites States, representing over 6% of the population. Diabetes is characterized by the inability to produce or properly use insulin. Type 2 diabetes (also called non-insulin-dependent diabetes or NIDDM) accounts for 80-90% of the diagnosed cases of diabetes and is caused by insulin resistance. Insulin resistance in type 2 diabetes prevents maintenance of blood glucose within desirable ranges, despite normal to elevated plasma levels of insulin.

Obesity is a major contributor to type 2 diabetes, as well as other illnesses including coronary heart disease, osteoarthritis, respiratory problems, and certain cancers. Despite attempts to control weight gain, obesity remains a serious health concern in the United States and other industrialized countries. Indeed, over 60% of adults in the United States are considered overweight, with about 22.5% of these being classified as obese.

With the high prevalence of diet-induced health concerns, such as diabetes and obesity, there remains a need for approaches that treat one or more of these conditions, including approaches with reduced side effects. Further, a better understanding of the mechanism of the products of phospholipid digestion is needed to facilitate such approaches. Overall, there is a need to develop methods, mechanisms and approaches for treating diet-induced conditions while limiting unwanted side effects.

### BRIEF SUMMARY OF THE INVENTION

One first aspect of the present invention provides methods of modulating lysophosphatidylcholine in a subject. Several approaches are contemplated for realizing this aspect of the invention.

In one approach, plasma lysophosphatidylcholine concentration or activity is modulated (e.g., reduced) in the subject.

In another approach, gastrointestinal lysophosphatidylcholine concentration is reduced in the subject. In some embodiments of this approach, lysophosphatidylcholine (LPC) concentration is reduced by selectively inhibiting phospholipase-A₂ in the gastrointestinal tract—without inhibiting or essentially not inhibiting one or more other enzymes that catalyze competing reactions involving the same substrate—specifically other enzymes that catabolize phosphatidylcholine (into reaction products other than LPC). For example, phospholipase-A₂ can be inhibited without inhibiting or essentially not inhibiting a gastrointestinal non-PLA₂ phospholipase having activity for hydrolysis of phosphatidylcholine (into reaction products other than lysophosphatidylcholine). As another example, phospholipase-A₂ can be inhibited without inhibiting or essentially not inhibiting a gastrointestinal lipase having activity for catabolizing phosphatidylcholine (into reaction products other than lysophosphatidylcholine). In additional embodiments of this approach, gastrointestinal LPC concentration is reduced by selectively enhancing enzymes that catalyze competing reactions involving the same substrate. In particular, for example, such embodiments can comprise increasing the concentration or activity of a gastrointestinal non-PLA₂ phospholipase having activity for catabolizing (e.g., via hydrolysis) of phosphatidylcholine (into reaction products other than lysophosphatidylcholine).

In a further approach, the concentration or activity of lysophosphatidylcholine can be modulated (e.g., reduced) by administering a lysophosphatidylcholine modulating agent that acts directly on lysophosphatidylcholine.

Combinations of these approaches, including various permutations thereof, are also contemplated in connection within this aspect of the invention.

Another second aspect of the invention provides methods for treating lysophosphatidylcholine-related conditions, generally, by modulating lysophosphatidylcholine activity and/or concentration in a subject. Preferably, lysophosphatidylcholine activity or concentration is modulated in a subject according to one or more of the approaches provided in connection with the first aspect of the invention. Some embodiments provide a method of treating a diet-induced condition by modulating lysophosphatidylcholine activity and/or concentration in a subject, preferably by reducing lysophosphatidylcholine activity and/or concentration. In some embodiments, the condition is an insulin-related condition, e.g., diabetes or diabetes type 2. In some embodiments, the condition is a weight-related condition, e.g., unwanted weight gain or obesity. In some embodiments, reduction is carried out by reduction of lysophospholipid production; in some embodimens, reduction is carried out by inhibiting phospholipase A2.

Another aspect of the present invention relates to lysophosphatidylcholine modulators that Can be used in the practice of the present invention. In some embodiments, the lysophophatidylcholine modulator is a phospholipase A2 inhibitor, and preferably, a selective phospholipase A2 inhibitor (e.g., as described in connection with the first aspect of the invention). Other lysophophatidylcholine modulators include agents that act on lysophosphatidylcholine, antibodies, and gene therapy approaches. The invention also relates to pharmaceutical compositions and kits comprising such lysophosphatidylcholine modulators for treatment of lysophosphatidylcholine-related conditions.

Those of skill in the art will recognize that the compounds described herein may exhibit the phenomena of tautomerism, conformational isomerism, geometric isomerism and/or optical isomerism. It should be understood that the invention encompasses any tautomeric, conformational isomeric, optical isomeric and/or geometric isomeric forms of the compounds having one or more of the utilities described herein, as well as mixtures of these various different forms. Prodrugs and active metabolites of the compounds described herein are also within the scope of the present invention.

### BRIEF DESCRIPTION OF THE DRAWINGS

FIG. 1 illustrates reduced absorption of lysophosphatidylcholine in phospholipase A2-deficient mice.

FIG. 2 illustrates an increase in insulin sensitivity in phospholipase A2-deficient mice.

FIG. 3 illustrates improvements in glucose tolerance in phospholipase A2-deficient mice.

FIG. 4 illustrates increases in glucose uptake by tissues in phospholipase A2-deficient mice.

FIG. 5 illustrates increased insulin-stimulated glucose metabolism under conditions of reduced levels of lysophosphatidylcholine in (a) HepG2 cells; (b) L6 myotube; and (c) 3T3L1 adipocytes.

FIG. 6 illustrates reduced post-prandial fat absorption in phospholipase A2-deficient mice on a high fat diet.

FIG. 7 illustrates decreased weight gain in phospholipase A2-deficient mice on a high fat compared with either (a) wild-type (+/+) or (b) heterozygous (+/−) mice.

FIG. 8 illustrates reduced weight gain in certain tissues of phospholipase A2-deficient mice on a high fat diet.

FIG. 9 illustrates reduced insulin and leptin levels in phospholipase A2-deficient mice fed a Western diet.

FIG. 10 illustrates no significant changes in body temperature and food intake in phospholipase A2-deficient mice.

FIG. 11 illustrates no lowering of phospholipid absorption in phospholipase A2-deficient mice.

FIG. 12 illustrates specificity of lysophosphatidylcholine reduction with respect to cholesterol absorption; FIG. 12(a) illustrates decreased cholesterol absorption produced by the phospholipase inhibitor, FPL 67047XX, whereas FIGS. 12(b) illustrates the lack of a decrease in cholesterol absorption in phospholipase A2-deficient mice fed a Western diet.

### DETAILED DESCRIPTION OF THE INVENTION

The present invention describes mechanisms useful in treating a lysophosphatidylcholine-related condition. Some embodiments of the invention involve modulating the production, absorption, and/or downstream activities of the products of phospholipase A2 (PLA2). In particular, the invention relates to reducing lysophosphatidylcholine (LPC) production and/or absorption in the gastrointestinal tract and/or reducing the activity of lysophosphatidylcholine. In some embodiments, LPC production and/or absorption can be inhibited by decreasing the activity of phospholipase A2. In some embodiments, the activity of LPC itself is reduced, e.g., by reducing the activity of lysophophatidylcholine in signaling pathways, and/or reducing its effectiveness as a signaling messenger. In some embodiments, a combination of both approaches can be used.

Such approaches find use, for example, in treating a lysophosphatidylcholine-related condition in which this metabolite plays a physiological role. Preferably, such approaches find use in treating a lysophosphatidylcholine-related condition that is induced by diet, including, for example, an insulin-related condition, e.g., diabetes or diabetes type 2, and/or a weight-related condition, e.g., unwanted weight gain or obesity, and other lysophosphatidylcholine-related conditions induced by diet, e.g., a high fat or Western diet.

Modulating lysophosphatidylcholine by any of the processes described herein provides methods of treating such conditions, described in detail below. Modulating lysophosphatidylcholine can include, for example and without limitation, changing (increasing or decreasing) concentration thereof, and/or changing (enhancing or inhibiting) activity (e.g., biological function) thereof.

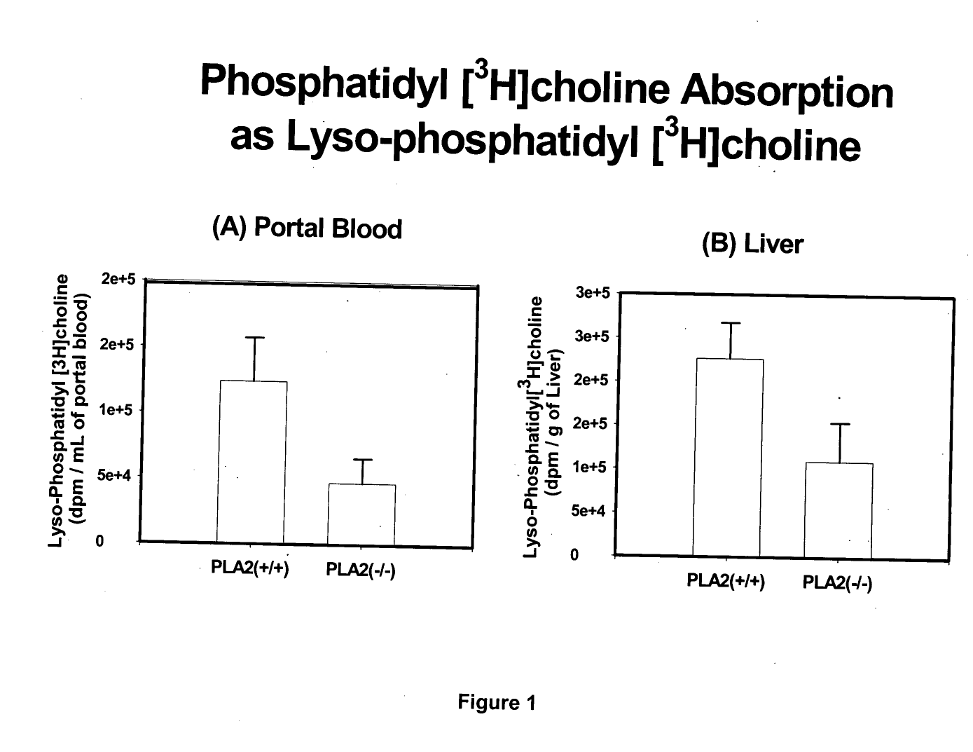

The effects of modulating lysophosphatidylcholine were evaluated in mice deficient in phospholipase A2. PLA2-deficient mice were generated by repeatedly back-crossing into the C57BL/6 background, as described in Huggins, et al., Protection against diet-induced obesity and obesity-related insulin resistance in Group 1B PLA2-deficient mice, Am. J. Physiol. Endocrinol. Metab. 283:E994-E1001, 2002, incorporated herein by reference, as well as in Richmond et al., Compensatory phospholipid digestion is required for cholesterol absorption in pancreatic phosphoipase A2-deficient mice, Gastroenterology, 120:1193-1202, 2001, also incorporated herein by reference. Absorption of lysophosphatidylcholine was reduced in PLA2-deficient mice. See, for example, FIG. 1. Thus, such mice can model conditions of reduced lysophosphatidylcholine.

Effects of Lysophosphatidylcholine with Respect to Diabetes

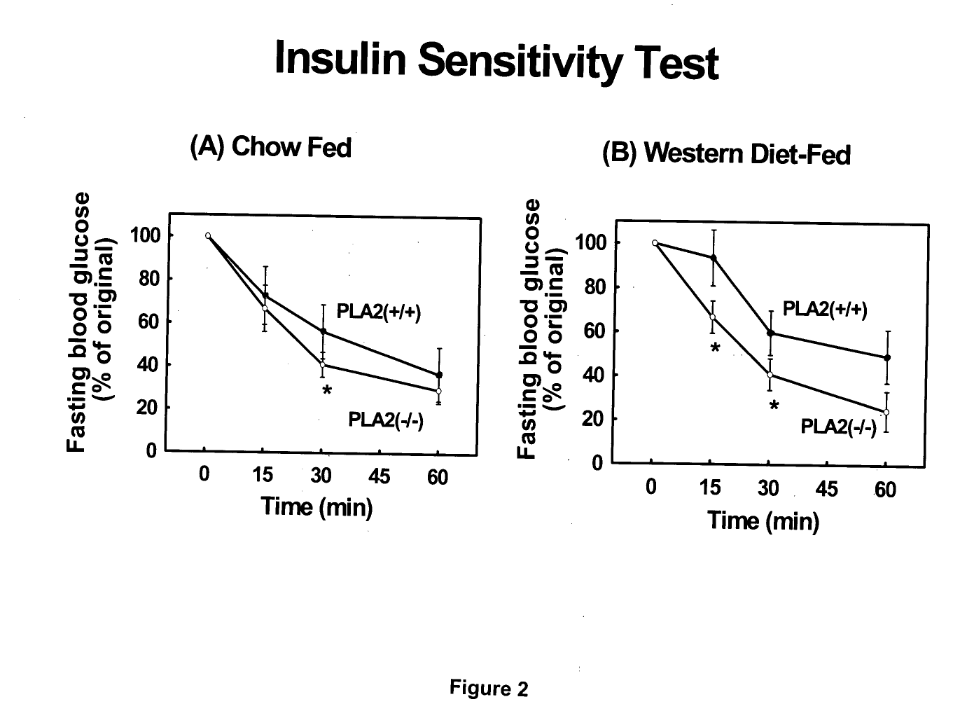

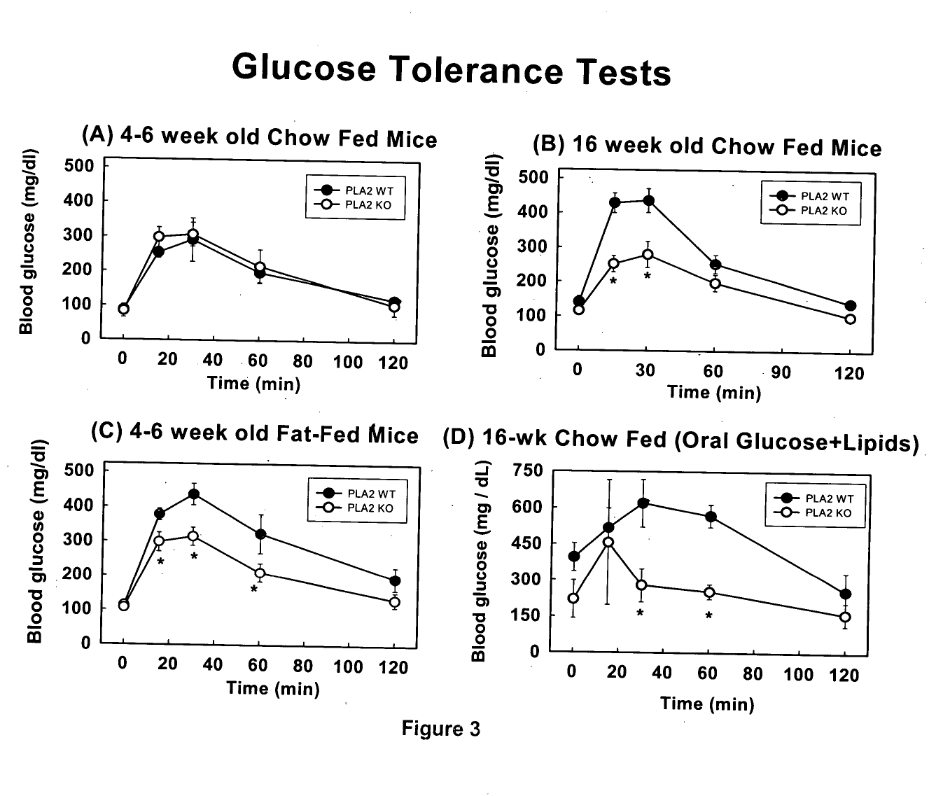

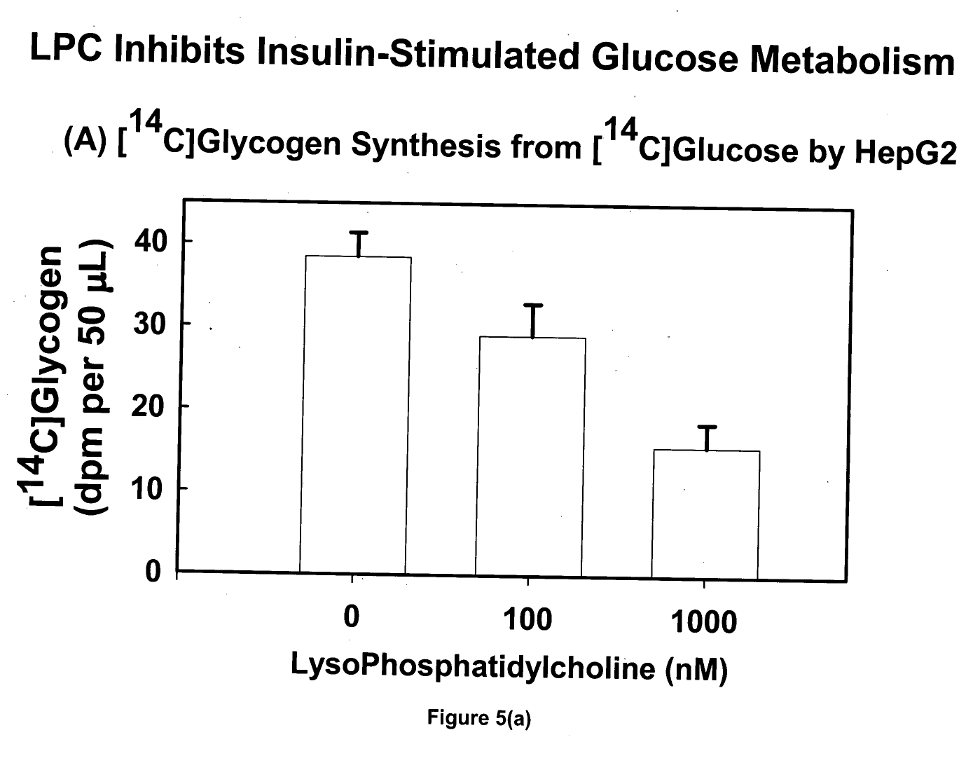

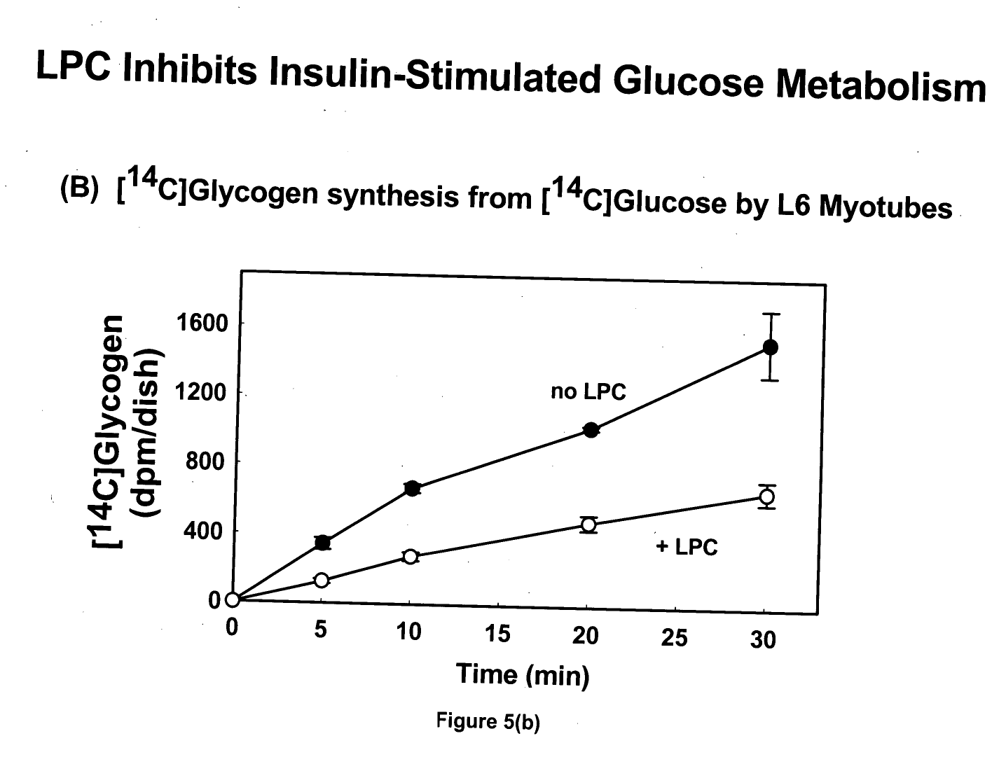

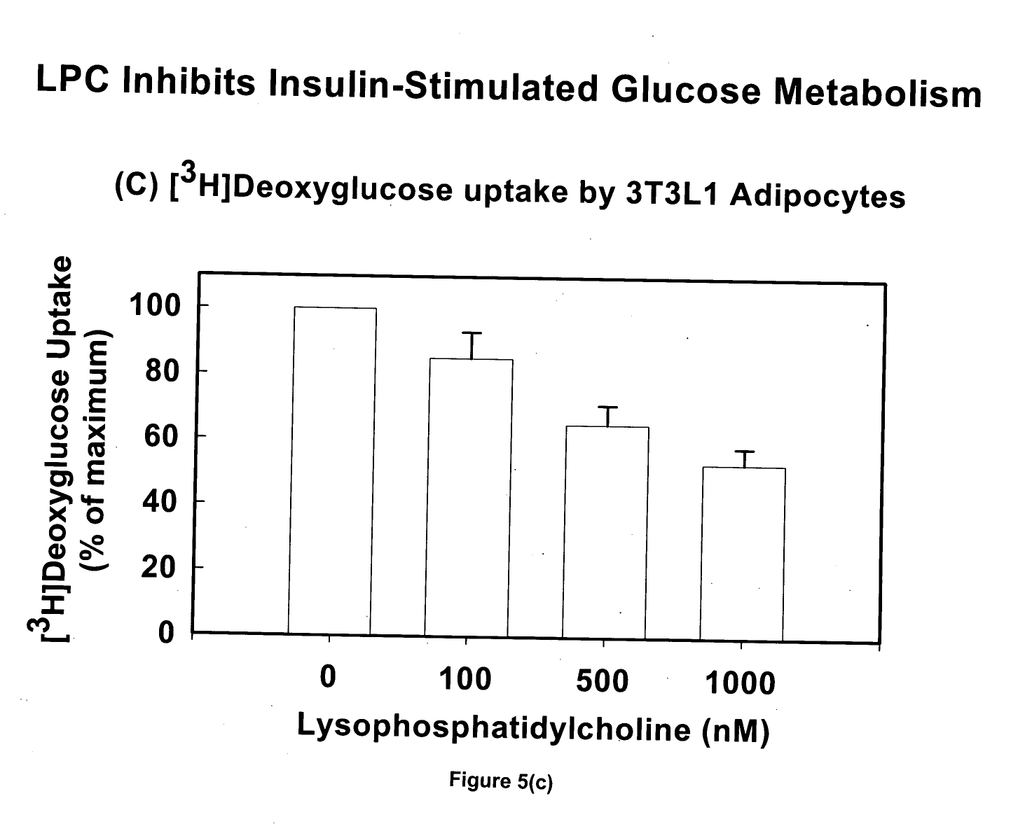

It has been observed that insulin sensitivity is increased in PLA2-deficient mice. See, for example, FIG. 2. Without being limited to a given hypothesis, such mice do not produce pancreatic phospholipase A2 and thus produce less lysophosphatidylcholine during digestion of phospholipids in the gut lumen. Also, glucose tolerance is improved under conditions of phospholipase A2 deficiency. See, for example, FIG. 3. Glucose uptake by tissues is increased in phospholipase A2-deficient mice. See, for example 4. Furthermore, insulin-stimulated glucose metabolism is increased under conditions of reduced levels of lysophosphatidylcholine in (a) HepG2 cells; (b) L6 myotube; and (c) 3T3L1 adipocytes. See, for example, FIG. 5.

In one aspect of the invention, phospholipase A2 and/or lysophosphatidylcholine is modulated to treat insulin-related conditions, including diabetes. In certain embodiments, reducing the amount of lysophosphatidylcholine in a patient produces a benefit in treating diabetes type 2. Such benefits include, but are not limited to, increased insulin-sensitivity, improved glucose tolerance, increased tissue glucose levels and tissue glucose metabolism, and/or increased insulin-stimulated glucose metabolism, for example, increased insulin-stimulated glucose metabolism in liver cells, skeletal muscle cells and/or adipocytes. Other benefits can include, but are not limited to, decreased post-prandial blood glucose levels, decreased fasting blood glucose levels, decreased fasting blood insulin levels, e.g., decreased fasting blood insulin levels in a subject resistant to insulin.

In some embodiments of the invention, the activity of phospholipase A2 can be inhibited. Other embodiments encompass the inhibition of the activity of lysophosphatidylcholine, e.g., reducing the activity of lysophophatidylcholine in signaling pathways, and/or reducing its effectiveness as a signaling messenger. In some embodiments, a combination of both approaches can be used.

Effects of Lysophosphatidylcholine with Respect to Weight Gain

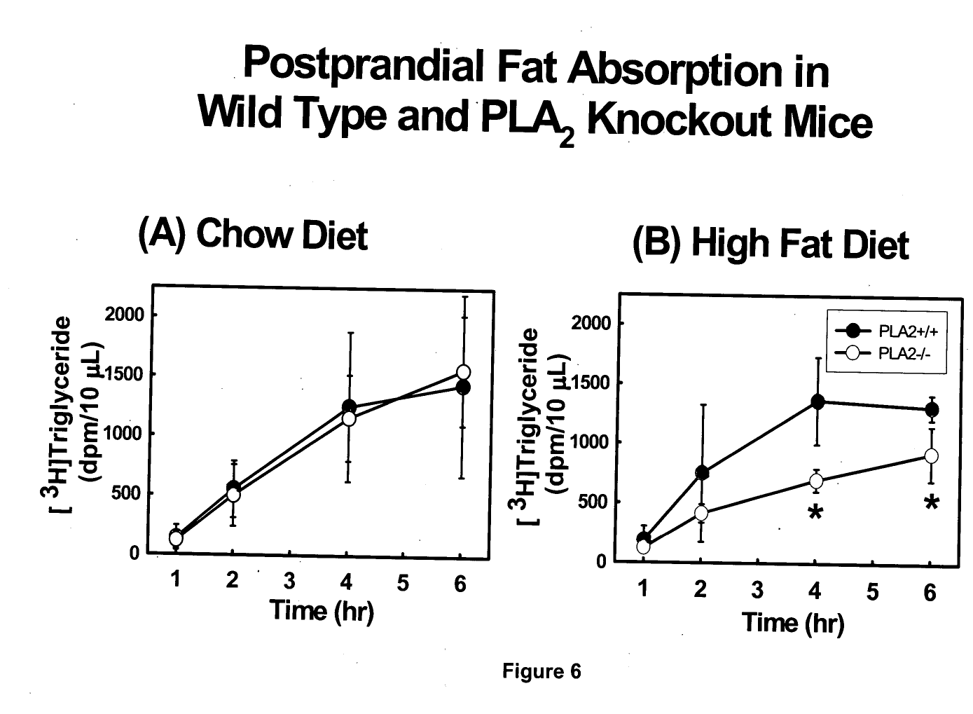

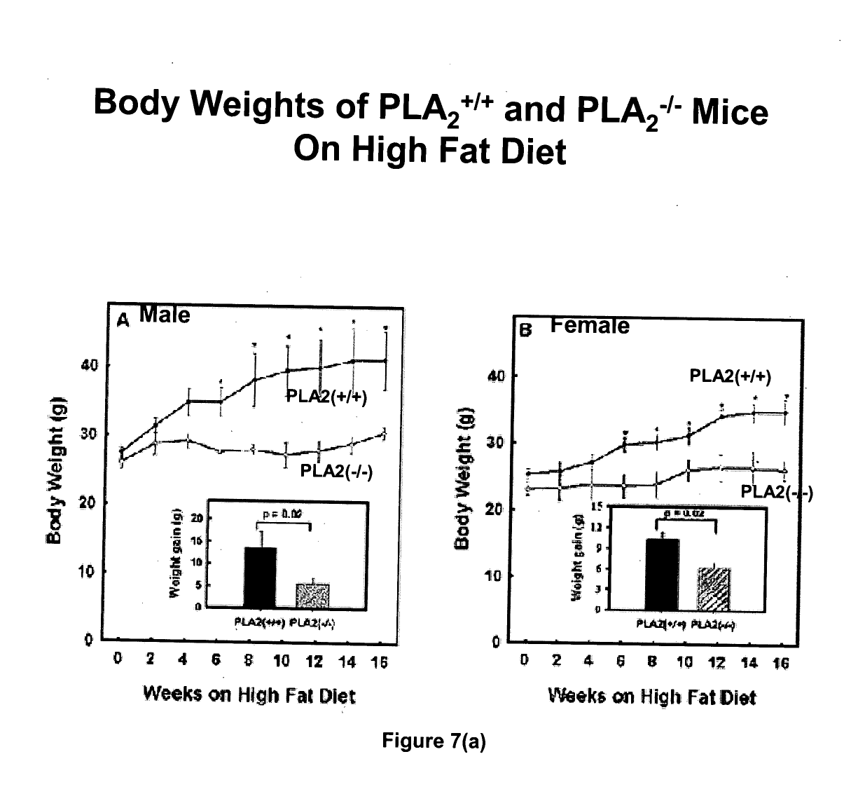

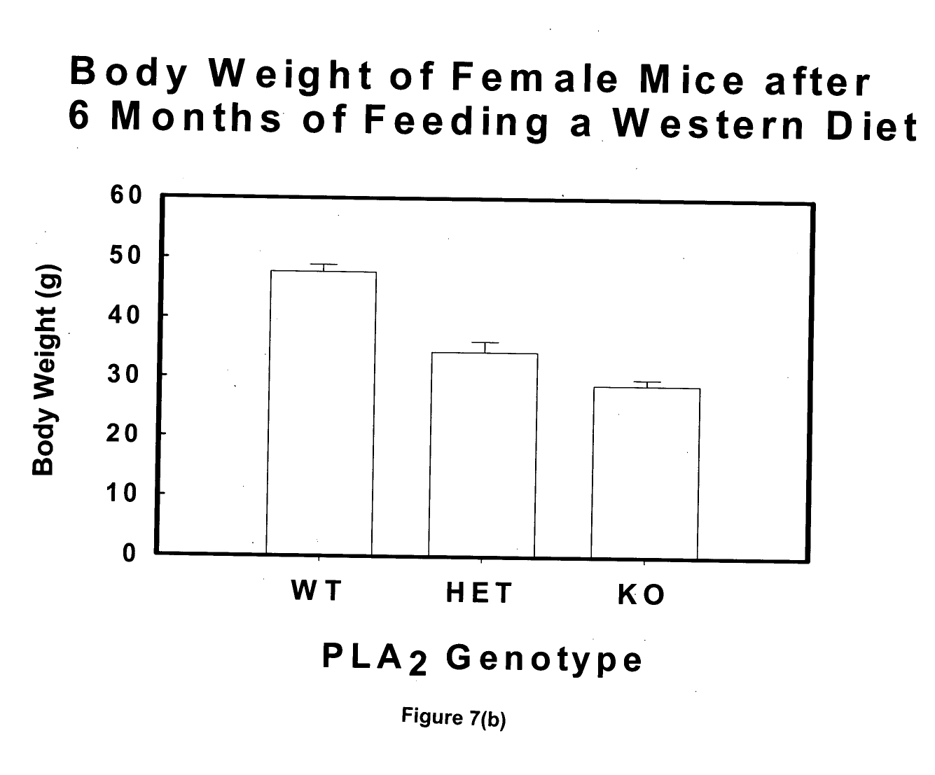

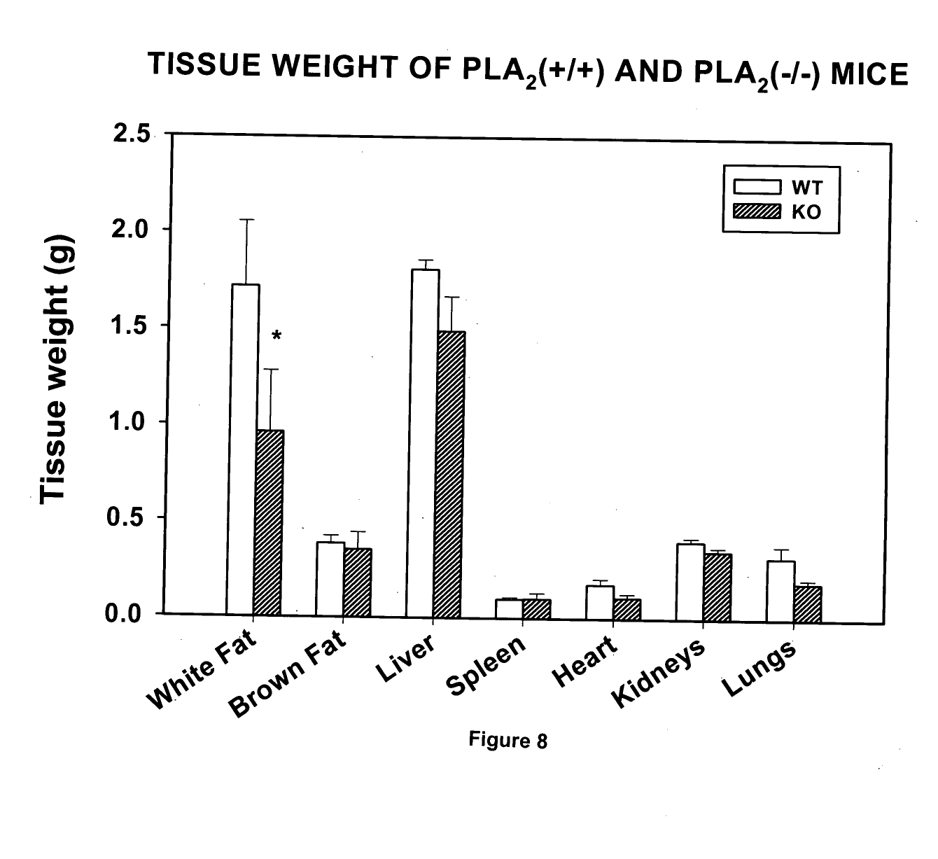

Post-prandial fat absorption is reduced in phospholipase A2-deficient mice on a high fat diet. See, for example, FIG. 6. Weight gain in phospholipase A2-deficient mice is reduced on a high fat diet compared with either (a) wild-type (+/+) or (b) heterozygous (+/−) mice. See, for example, FIG. 7. Weight gain in certain tissues is reduced in phospholipase A2-deficient mice. See, for example, FIG. 8.

In another aspect of the invention, phospholipase A2 and/or lysophosphatidylcholine is modulated to treat weight-related conditions, including obesity. In certain embodiments, reducing lysophosphatidylcholine production and/or activity, for example as taught in the present invention, decreases fat absorption and/or reduces weight gain.

In some embodiments of the invention, the activity of phospholipase A2 can be inhibited. Other embodiments encompass the inhibition of the activity of lysophosphatidylcholine itself, e.g., reducing the activity of lysophophatidylcholine in signaling pathways, and/or reducing its effectiveness as a signaling messenger. In some embodiments, a combination of both approaches can be used. In certain embodiments, inhibiting phospholipase A2, reducing lysophosphatidylcholine and/or its activity can reduce weight gain in certain tissues and organs, e.g., white fat.

Effects of Lysophosphatidylcholine on Diabetes and Weight Gain

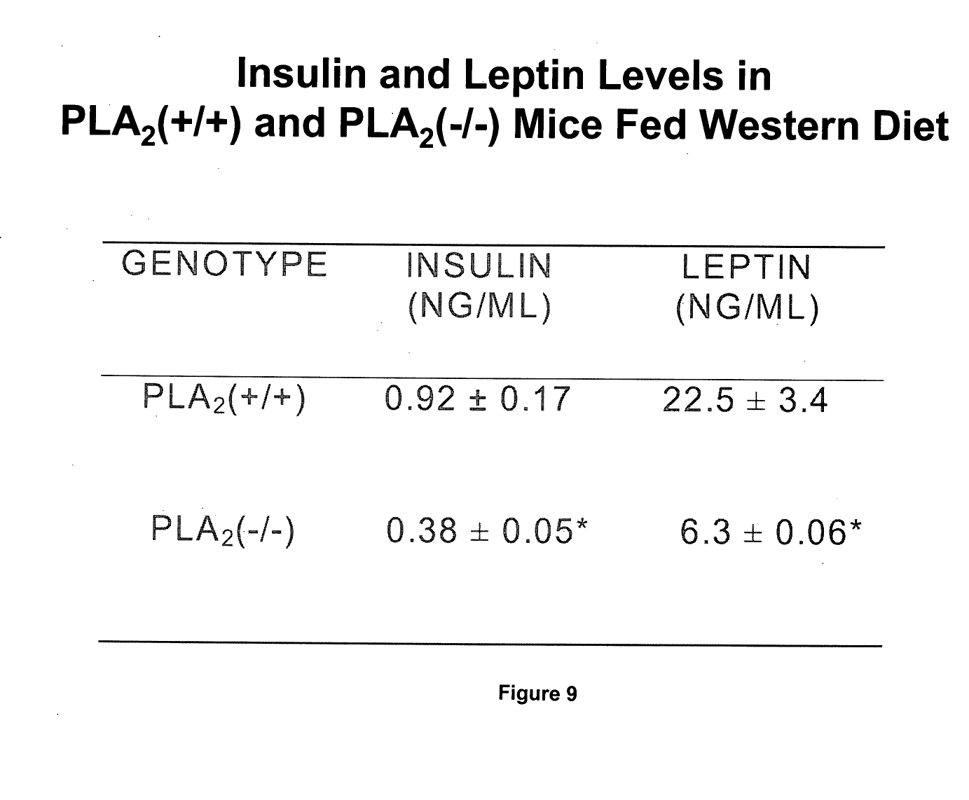

Insulin and leptin levels are reduced in phospholipase A2-deficient mice fed a Western diet. See, for example, FIG. 9.

Another aspect of the present invention provides methods of reducing or delaying the onset of diet-induced diabetes through weight gain. An unchecked high fat diet can produce not only unwanted weight gain, but also can contribute to diabetic insulin resistance. This resistance may be recognized by decreased insulin and leptin levels in a subject. Methods of modulating lysophosphatidylcholine and/or phospholipase A2 disclosed herein can be used in the prophylactic treatment of diet-induced diabetes.

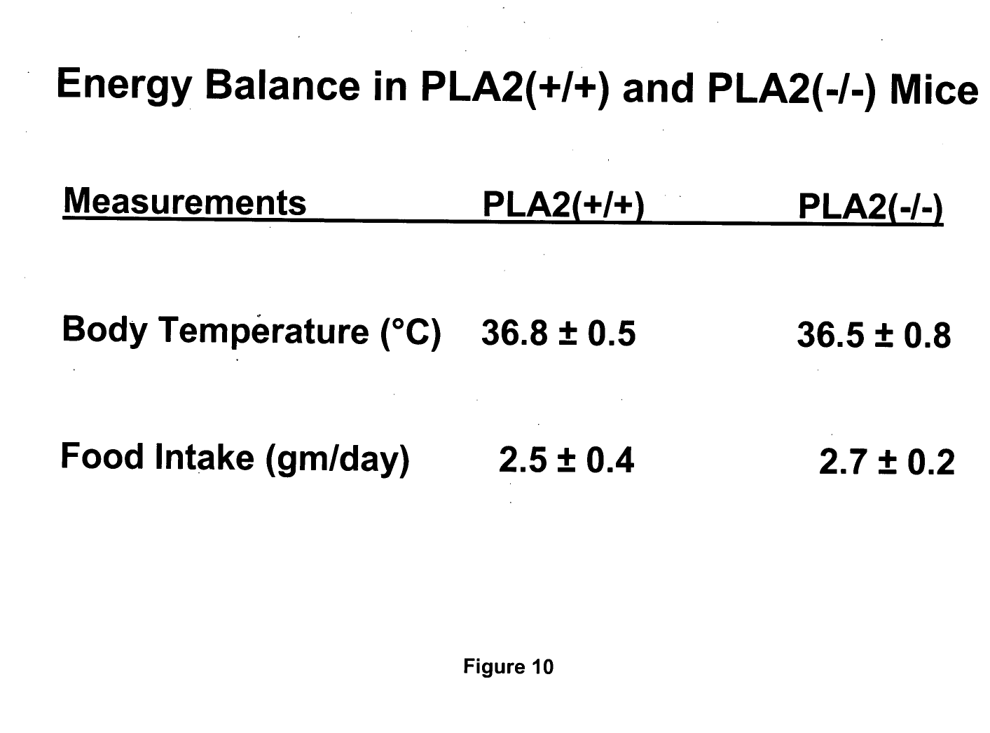

No significant changes in body temperature and food intake are observed in phospholipase A2-deficient mice. See, for example, FIG. 10.

Modulating lysophosphatidylcholine represents a novel approach to controlling lysophosphatidylcholine-related conditions, such as weight gain, as well as avoiding and treating other diet-induced conditions, such as diabetes. In some preferred embodiments, these effects can be realized without a change in diet and/or activity on the part of the subject. For example, modulating lysophosphatidylcholine levels and/or activity may reduce lysophosphatidylcholine so as to result in a decrease in fat absorption and/or a reduction in weight gain in a subject on a high fat diet compared to if the subject was not receiving Iysophosphatidylcholine-modulating treatment. More preferably, this decrease and/or reduction occurs without a significant change in energy expenditure and/or food intake of the subject, and without a significant change in the body temperature of the subject.

Fewer Side-Effects from Reducing Lysophosphatidylcholine in Treating Diet-Induced Conditions

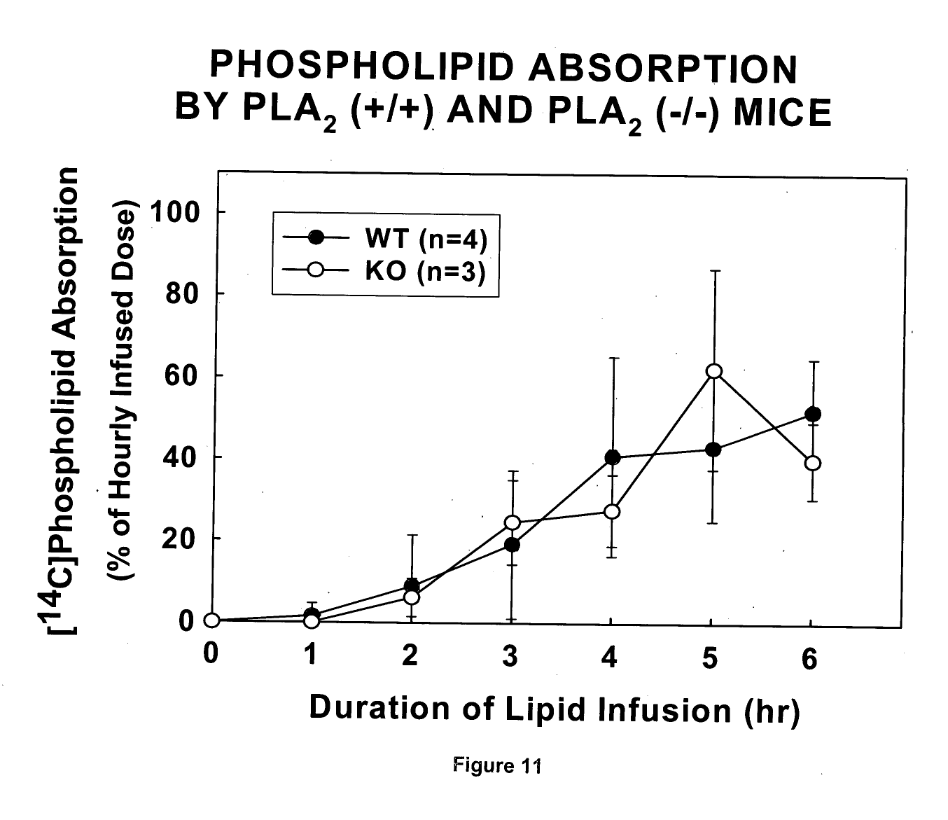

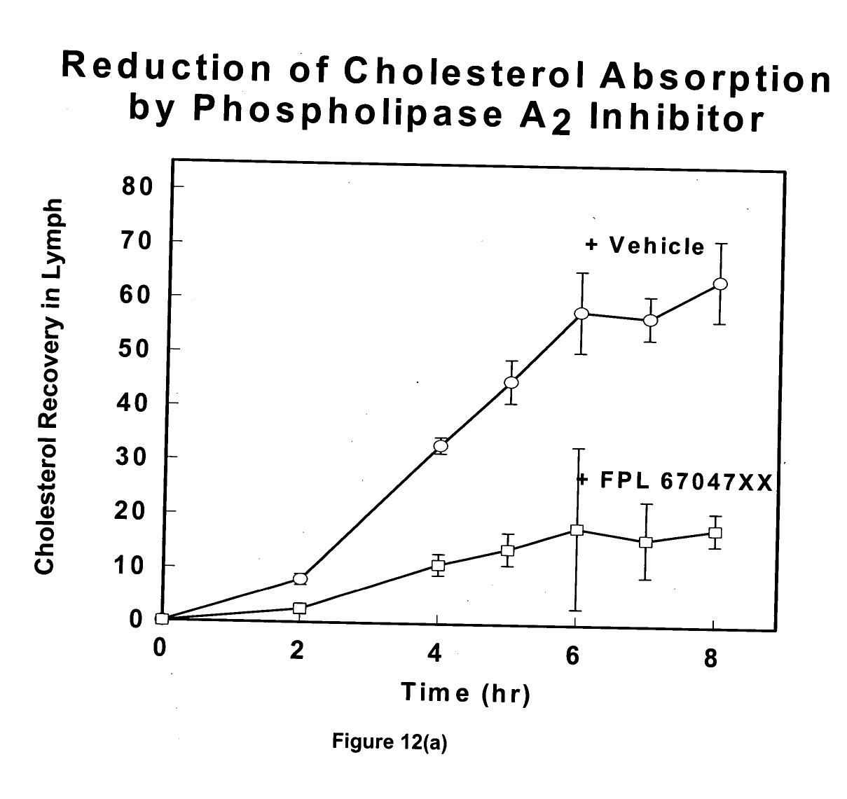

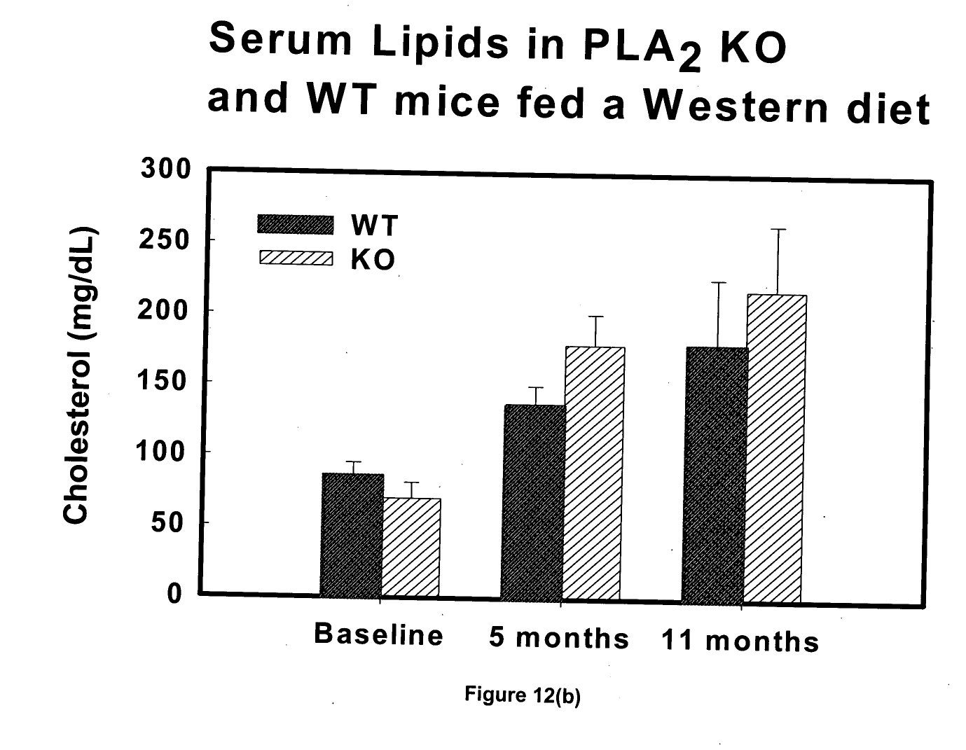

Phospholipid absorption is not lowered in phospholipase A2-deficient mice. See, for example, FIG. 11. FIG. 12 illustrates specificity of lysophosphatidylcholine reduction, with respect to cholesterol absorption; FIG. 12(a) illustrates decreased cholesterol absorption produced by the phospholipase inhibitor, FPL 67047XX, whereas FIG. 12(b) illustrates the lack of a decrease in cholesterol absorption in phospholipase A2-deficient mice fed a Western diet.

In preferred embodiments, phospholipase A2 and/or lysophosphatidylcholine is modulated to offset certain negative consequences of high fat diets without affecting normal aspects of metabolism on non-high fat diets. In some of these embodiments, the activity of phospholipase A2 can be inhibited. Other embodiments encompass the inhibition of the activity of lysophosphatidylcholine, e.g., reducing the activity of lysophophatidylcholine in signaling pathways, and/or reducing its effectiveness as a signaling messenger. In some embodiments, a combination of both approaches can be used. Further, in certain embodiments, reducing lysophosphatidylcholine levels and/or activity does not affect cholesterol absorption, or cholesterol absorption efficiency, of a subject receiving LPC-modulating treatment, for example, when the subject is on a high fat diet. In certain embodiment, reducing LPC levels and/or activity results in no significant lowering of phospholipid absorption. Inhibition of phospholipase A2 may also have little or no effect, preferably no effect or essentially no effect, on fat absorption or on the absorption of fat-soluble vitamins.

Methods of Treating Diet-Induced Conditions

Another aspect of the present invention relates to methods, mechanisms, and approaches for treating a diet-induced condition by modulating lysophosphatidylcholine, for example, reducing lysophosphatidylcholine plasma concentration by a therapeutic amount in a subject. Therapeutic modulation, e.g., therapeutic reduction, of this metabolite can be achieved in a number of ways, including, for example, reducing lysophosphatidylcholine production in the gastrointestinal tract, as well as by reducing the activity of lysophosphatidylcholine. In some embodiments, for example, the activity of phospholipase A2 is reduced, e.g. using a phospholipase A2 inhibitor.

The present invention provides methods, mechanisms, and approaches for the treatment of animal subjects. The term “animal subject” as used herein includes humans as well as other mammals. The term “treating” as used herein includes achieving a therapeutic benefit and/or a prophylactic benefit. By therapeutic benefit is meant eradication or amelioration of the underlying disorder being treated. For example, in a diabetic patient, therapeutic benefit includes eradication or amelioration of the underlying diabetes. Also, a therapeutic benefit is achieved with the eradication or amelioration of one or more of the physiological symptoms associated with the underlying disorder such that an improvement is observed in the patient, notwithstanding the fact that the patient may still be afflicted with the underlying disorder. For example, reducing lysophosphatidylcholine can provide therapeutic benefit not only when insulin resistance is corrected, but also when an improvement is observed in the patient with respect to other disorders that accompany diabetes like fatigue, blurred vision, or tingling sensations in the hands or feet. For prophylactic benefit, lysophosphatidylcholine may be reduced in a patient at risk of developing a diet-induced condition, e.g., diabetes or obesity, or to a patient reporting one or more of the physiological symptoms of such conditions, even though a diagnosis of diabetes and/or obesity may not have been made.

The methods for therapeutically modulating lysophosphatidylcholine described herein can apply to any lysophosphatidylcholine-related condition, that is, to any condition in which this metabolite plays a physiological role. Preferably, such conditions include lysophosphatidylcholine-related conditions induced by diet, that is, conditions in which lysophosphatidylcholine plays a physiological role and which are brought on, accelerated, exacerbated, or otherwise influenced by diet. Diet-induced conditions include, but are not limited to, insulin-related conditions, e.g., diabetes and diabetes type 2, and weight-related conditions, e.g., unwanted weight gain and obesity, as well as hyperlipidemia, hypercholesterolemia, cardiovascular disease, such as heart disease and stroke, hypertension, cancer, sleep apnea, and osteoarthritis, gallbladder disease, fatty liver disease, and the like. In particular, lysophosphatidylcholine-related conditions induced by diet include unwanted weight gain and/or diabetes type 2, produced as a result of consumption of a high fat or Western diet.

Western Diets and Western-Related Diets

Generally, some embodiments of the invention relate to one or more of a high-carbohydrate diet, a high-saccharide diet, a high-fat diet and/or a high-cholesterol diet, in various combinations. Such diets are generally referred to herein as a “high-risk diets” (and can include, for example, Western diets). Such diets can heighten the risk profile of a subject patient for one or more conditions, including an obesity-related condition, an insulin-related condition and/or a cholesterol-related condition. In particular, such high-risk diets can, in some embodiments, include at least a high-carbohydrate diet together with one or more of a high-saccharide diet, a high-fat diet and/or a high-cholesterol diet. A high-risk diet can also include a high-saccharide diet in combination with one or both of a high-fat diet and/or a high-cholesterol diet. A high-risk diet can also comprise a high-fat diet in combination with a high-cholesterol diet. In some embodiments, a high-risk diet can include the combination of a high-carbohydrate diet, a high-saccharide diet and a high-fat diet. In other embodiments, a high-risk diet can include a high-carbohydrate diet, a high-saccharide diet, and a high-cholesterol diet. In other embodiments, a high-risk diet can include a high-carbohydrate diet, a high-fat diet and a high-cholesterol diet. In yet further embodiments, a high-risk diet can include a high-saccharide diet, a high-fat diet and a high-cholesterol diet. In some embodiments, a high-risk diet can include a high-carbohydrate diet, a high-saccharide diet, a high-fat diet and a high-cholesterol diet.

Generally, the diet of a subject can comprise a total caloric content, for example, a total daily caloric content. In some embodiments, the subject diet can be a high-fat diet. In such embodiments, at least about 50% of the total caloric content can come from fat. In other such embodiments, at least about 40%, or at least about 30% or at least about 25%, or at least about 20% of the total caloric content can come from fat. In some embodiments, in which a high-fat diet is combined with one or more of a high-carbohydrate diet, a high-saccharide diet or a high-cholesterol diet, at least about 15% or at least about 10% of the total caloric content can come from fat.

Similarly, in some embodiments, the diet can be a high-carbohydrate diet. In such embodiments, at least about 50% of the total caloric content can come from carbohydrates. In other such embodiments, at least about 40%, or at least about 30% or at least about 25%, or at least about 20% of the total caloric content can come from carbohydrates. In some embodiments, in which a high-carbohydrate diet is combined with one or more of a high-fat diet, a high-saccharide diet or a high-cholesterol diet, at least about 15% or at least about 10% of the total caloric content can come from carbohydrate.

Further, in some embodiments, the diet can be a high-saccharide diet. In embodiments; at least about 50% of the total caloric content can come from saccharides. In other such embodiments, at least about 40%, or at least about 30% or at least about 25%, or at least about 20% of the total caloric content can come from saccharides. In some embodiments, in which a high-saccharide diet is combined with one or more of a high-fat diet, a high-carbohydrate diet or a high-cholesterol diet, at least about 15% or at least about 10% of the total caloric content can come from saccharides.

Similarly, in some embodiments, the diet can be a high-cholesterol diet. In such embodiments, the diet can comprise at least about 1% cholesterol (wt/wt, relative to fat). In other such embodiments, the diet can comprise at least about 0.5% or at least about 0.3% or at least about 0.1%, or at least about 0.07% cholesterol (wt/wt relative to fat). In some embodiments, in which a high-cholesterol diet is combined with one or more of a high-fat diet, a high-carbohydrate diet or a high-saccharide diet, the diet can comprise at least about 0.05% or at least about 0.03% cholesterol (wt/wt, relative to fat).

As an example, a high fat diet can include, for example, diets high in meat, dairy products, and alcohol, as well as possibly including processed food stuffs, red meats, soda, sweets, refined grains, deserts, and high-fat dairy products, for example, where at least about 25% of calories come from fat and at least about 8% come from saturated fat; or at least about 30% of calories come from fat and at least about 10% come from saturated fat; or where at least about 34% of calories came from fat and at least about 12% come from saturated fat; or where at least about 42% of calories come from fat and at least about 15% come from saturated fat; or where at least about 50% of calories come from fat and at least about 20% come from saturated fat. One such high fat diet is a “Western diet” which refers to the diet of industrialized countries, including, for example, a typical American diet, Western European diet, Australian diet, and/or Japanese diet. One particular example of a Western diet comprises at least about 17% fat and at least about 0.1% cholesterol (wt/wt); at least about 21% fat and at least about 0.15% cholesterol (wt/wt); or at least about 25% and at least about 0.2% cholesterol (wt/wt).

Such high-risk diets may include one or more high-risk foodstuffs.

Considered in the context of a foodstuff, generally, some embodiments of the invention relate to one or more of a high-carbohydrate foodstuff, a high-saccharide foodstuff, a high-fat foodstuff and/or a high-cholesterol foodstuff, in various combinations. Such foodstuffs are generally referred to herein as a “high-risk foodstuffs” (including for example Western foodstuffs). Such foodstuffs can heighten the risk profile of a subject patient for one or more conditions, including an obesity-related condition, an insulin-related condition and/or a cholesterol-related condition. In particular, such high-risk foodstuffs can, in some embodiments, include at least a high-carbohydrate foodstuff together with one or more of a high-saccharide foodstuff, a high-fat foodstuff and/or a high-cholesterol foodstuff. A high-risk foodstuff can also include a high-saccharide foodstuff in combination with one or both of a high-fat foodstuff and/or a high-cholesterol foodstuff. A high-risk foodstuff can also comprise a high-fat foodstuff in combination with a high-cholesterol foodstuff. In some embodiments, a high-risk foodstuff can include the combination of a high-carbohydrate foodstuff, a high-saccharide foodstuff and a high-fat foodstuff. In other embodiments, a high-risk foodstuff can include a high-carbohydrate foodstuff, a high-saccharide foodstuff, and a high-cholesterol foodstuff. In other embodiments, a high-risk foodstuff can include a high-carbohydrate foodstuff, a high-fat foodstuff and a high-cholesterol foodstuff. In yet further embodiments, a high-risk foodstuff can include a high-saccharide foodstuff, a high-fat foodstuff and a high-cholesterol foodstuff. In some embodiments, a high-risk foodstuff can include a high-carbohydrate foodstuff, a high-saccharide foodstuff, a high-fat foodstuff and a high-cholesterol foodstuff.

Hence, the food product composition can comprise a foodstuff having a total caloric content. In some embodiments, the food-stuff can be a high-fat foodstuff. In such embodiments, at least about 50% of the total caloric content can come from fat. In other such embodiments, at least about 40%, or at least about 30% or at least about 25%, or at least about 20% of the total caloric content can come from fat. In some embodiments, in which a high-fat foodstuff is combined with one or more of a high-carbohydrate foodstuff, a high-saccharide foodstuff or a high-cholesterol foodstuff, at least about 15% or at least about 10% of the total caloric content can come from fat.

Similarly, in some embodiments, the food-stuff can be a high-carbohydrate foodstuff. In such embodiments, at least about 50% of the total caloric content can come from carbohydrates. In other such embodiments, at least about 40%, or at least about 30% or at least about 25%, or at least about 20% of the total caloric content can come from carbohydrates. In some embodiments, in which a high-carbohydrate foodstuff is combined with one or more of a high-fat foodstuff, a high-saccharide foodstuff or a high-cholesterol foodstuff, at least about 15% or at least about 10% of the total caloric content can come from carbohydrate.

Further, in some embodiments, the food-stuff can be a high-saccharide foodstuff. In such embodiments, at least about 50% of the total caloric content can come from saccharides. In other such embodiments, at least about 40%, or at least about 30% or at least about 25%, or at least about 20% of the total caloric content can come from saccharides. In some embodiments, in which a high-saccharide foodstuff is combined with one or more of a high-fat foodstuff, a high-carbohydrate foodstuff or a high-cholesterol foodstuff, at least about 15% or at least about 10% of the total caloric content can come from saccharides.

Similarly, in some embodiments, the food-stuff can be a high-cholesterol foodstuff. In such embodiments, the food-stuff can comprise at least about 1% cholesterol (wt/wt, relative to fat). In other such embodiments, the foodstuff can comprise at least about 0.5%, or at least about 0.3% or at least about 0.1%, or at least about 0.07% cholesterol (wt/wt relative to fat). In some embodiments, in which a high-cholesterol foodstuff is combined with one or more of a high-fat foodstuff, a high-carbohydrate foodstuff or a high-saccharide foodstuff, the foodstuff can comprise at least about 0.05% or at least about 0.03% cholesterol (wt/wt, relative to fat).

As noted above, the methods of the invention can be used advantageously together with other methods, including for example methods broadly directed to treating insulin-related conditions, weight-related conditions and/or cholesterol-related conditions (including dislipidemia generally) and any combination thereof. Aspects of such conditions are described below.

Modulators of Lysophosphatidylcholine

Generally, in some embodiments, lysophosphatidylcholine can be modulated using compounds effective, for example, for bring about a reduction in lysophosphatidylcholine production, absorption, concentration and/or activity. Such compounds form the basis of pharmaceutical compositions and kits that find use in methods of treating a subject by administering the composition.

Preferably, plasma lysophosphatidylcholine concentration or activity is modulated (e.g., reduced) in the subject. Plasma LPC concentration or activity can be modulated directly or indirectly in the plasma. Alternatively, plasma LPC can be effectively modulated by reducing the concentration of gastrointestinal LPC and additionally or alternatively by reducing the absorbtion of LPC from the gastrointestinal tract into system circulation. The gastrointestinal concentration of LPC and/or the level of absorbtion of LPC can, in turn, be modulated directly or indirectly.

In some embodiments, the activity of lysophosphatidylcholine itself can be modulated e.g., to interfere with the signaling effects of this metabolite, e.g., by reducing the activity of lysophophatidylcholine in signaling pathways, and/or reducing its effectiveness as a signaling messenger. In some embodiments, for instance, compounds may be used that bring about hydrolysis of lysophosphatidylcholine into forms that do not play the same role as lysophosphatidylcholine in signaling pathways. In still other embodiments, a combination of both approaches can be used, where both the amount and activity of lysophosphatidylcholine can be modulated, e.g., reduced.

For example, one or more LPC modulating agents can be used to directly modulate LPC. Such direct modulating agents can include, for example, enzymes having activity for catabolizing LPC (e.g., such as lysophospholipases), and non-enzymatic agents. Generally, such modulating agents can comprise (or can consist essentially of) one or more of small substituted organic molecules, oligomers, polymers, oligomer moieties, polymer moieties, LPC-binding moieties, and combinations thereof (e.g., including an LPC inhibitor comprising an LPC-binding moiety covalently linked to a non-absorbable or non-absorbed moiety, such as an oligomer moiety or a polymer moiety.

In other embodiments, one or more LPC modulating agents can indirectly modulate LPC. In one approach, for example, compositions comprising a LPC modulating agent can reduce production of lysophosphatidylcholine, for example by reducing the activity of phospholipase A2 and/or one or more other phospholipases. Preferably, in some embodiments, LPC concentration is reduced by selectively inhibiting phospholipase-A₂ in the gastrointestinal tract—without inhibiting or essentially not inhibiting one or more other enzymes that catalyze competing reactions involving the same substrate—specifically other enzymes that catabolize phosphatidylcholine (into reaction products other than LPC). For example, phospholipase-A₂ can be inhibited without inhibiting or essentially not inhibiting a gastrointestinal non-PLA₂ phospholipase having activity for hydrolysis of phosphatidylcholine (into reaction products other than lysophosphatidylcholine). As another example, phospholipase-A₂ can be inhibited without inhibiting or essentially not inhibiting a gastrointestinal lipase having activity for catabolizing phosphatidylcholine (into reaction products other than lysophosphatidylcholine).

As a further example, gastrointestinal LPC concentration can be modulated (e.g., reduced) by enhancing, and preferably selectively enhancing, enzymes that catalyze competing reactions involving the same substrate. In particular, for example, such embodiments can comprise increasing the concentration or activity of a gastrointestinal non-PLA₂ phospholipase having activity for catabolizing (e.g., via hydrolysis) of phosphatidylcholine (into reaction products other than lysophosphatidylcholine). In some embodiments, for example, a phospholipase inhibitor is used that inhibits phospholipase A2 but does not inhibit or essentially does not inhibit phospholipase B. In some embodiments, for example, a phospholipase inhibitor is used that inhibits phospholipase A2 but does not inhibit or essentially does not inhibit one or more of phospholipase C or phospholipase D. In some embodiments, the phospholipase inhibitor inhibits phospholipase A2 but does not inhibit or essentially does not inhibit any other gastrointestinal phospholipases, including phospholipase A1 and phospholipase B, or including each of phospholipase A, phospholipase B, phospholipase C and phospholipase D. In some embodiments, the phospholipase inhibitor inhibits phospholipase A2 but does not inhibit or essentially does not inhibit any other gastrointestinal lipases, e.g., carboxyl ester lipase and pancreatic triglyceride lipase. In some embodiments, a phospholipase inhibitor is used that inhibits phospholipase A2 to a greater extent than to other phospholipases and/or lipases.

Generally, the term “inhibits” and its grammatical variations are not intended to require a complete inhibition of enzymatic activity. For example, it can refer to a reduction in enzymatic activity by at least about 50%, at least about 75%, preferably by at least about 90%, more preferably by at least about 98%, and even more preferably by at least about 99% of the activity of the enzyme in the absence of the inhibitor. Most preferably, it refers to a reduction in enzyme activity by an effective amount, that is by an amount sufficient to produce a therapeutic and/or prophylactic benefit in at least one condition being treated in a subject receiving LPC-modulating treatment, e.g., as disclosed herein. Conversely, the phrase “does not inhibit” and its grammatical variations refers to situations where there is less than about 20%, less than about 10%, less than about 5%, preferably less than about 2%, and more preferably less than about 1% of reduction in enzyme activity in the presence of the inhibitor. Most preferably, it refers to a reduction in enzyme activity that is not sufficient to produce a noticeable effect in a patient receiving treatment. Further, the phrase “essentially does not inhibit” and its grammatical variations refers to situations where there is less than about 30%, less than about 25%, less than about 20%, preferably less than about 15%, and more preferably less than about 10% of reduction in enzyme activity in the presence of the inhibitor.

Phospholipase-A₂ inhibitors are well known in the art. For example, small molecule inhibitors of phospholipases can be used, preferably inhibitors of phospholipase A2, such as FPL 67047XX and/or MJ99, to reduce lysophosphatidylcholine production. Other phospholipase inhibitors useful in the practice of the methods of this invention include arachidonic acid analogues (e.g., arachidonyl trifluoromethyl ketone, methylarachidonyl fluorophosphonate, and palmitoyl trifluoromethyl ketone), benzensulfonamide derivatives, bromoenol lactone, p-bromophenyl bromide, bromophenacyl bromide, trifluoromethylketone, sialoglycolipids, proteoglycans, and the like, as well as phospholipase A2 inhibitors disclosed in WO 03/101487, incorporated herein by reference.

In some embodiments, reduction of lysophosphatidylcholine can be achieved by indirectly inhibiting the activity of phospholipase A2, for example, by acting against substances that activate phosholipase A2. For example, trypsin and bile salts play roles in activating phospholipase A2 digestion. Down regulating this activation by reducing production and/or activity of trypsin and/or bile acids can reduce activity of phospholipase A2, resulting in reduced lysophosphatidylcholine. In some embodiments, co-enzymes that aid phoshpholipase A2 digestion can be inhibited or otherwise modified to reduce co-enzymatic activity with respect to phospholipase A2.

In some embodiments, lysophosphatidylcholine can be modulated (directly or indirectly) using antibodies. For example, antibodies to phospholipase A2, or fragments thereof, such as Fab fragments, can be used to decrease phospholipase A2 activity, with the effect of reducing the production of lysophosphatidylcholine. Also, antibodies to any of the substances involved in activation of phospholipase A2 and/or involved in co-enzymatic activity of phospholipase A2 can also be used.

Further, in some embodiments, the activity of phospholipase A2 can be modulated (directly or indirectly) using gene therapy approaches. Gene therapy techniques for reducing transcription, translation, and/or activity of a gene product are known in the art and can be applied to phospholipases, preferably phospholipase A2. Such techniques include, for example, antisense and/or siRNA, for example, using a composition comprising an antisense nucleic acid complementary to the phospholipase A2 gene, or an siRNA or siRNA-like molecule that silences or reduces phospholipase A2 expression. Silencing gene therapy approaches can also be used against any of the substances involved in activation of phospholipase A2 and/or co-enzymatic activity of phospholipase A2. Alternatively, increasing the production of non-PLA2 phospholipases, e.g., phospholipase A1 or phospholipase B can bring about hydrolysis of phosphatidylcholine to products other than lysophosphatidylcholine. For example, phospholipase B digests phosphatidylcholine to form glycerol 3-phosphorylcholine instead.

Enzymes that catalyze reactions competing with PLA2 hydrolysis of phosphatidylcholine are known in the art. For example, phosphatidylcholine hydrolysis can be catalyzed by one or more of phospholipase D (PLD), phospholipase C (PLC), phospholipase B (PLB), phospholipase A1 (PLA1) as well as by phospholipase A2 (PLA2). Enzymes that modulate LPC directly, such as lysophospholipases, are also known in the art.

Various pathways for phosphatidylcholine hydrolysis by these phospholipase enzymes, PLD, PLC, PLA2, PLB and PLA1 is represented schematically below: 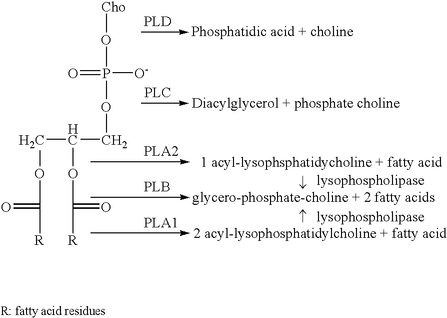
In the above schematic representation, the large (left-to-right) arrows lines generally indicate the site of cleavage for the associated phospholipases. For the lysophospholipase enzymes, the site of cleavage is either the 1-acyl or 2-acyl group of lysophosphatidylcholine.

Phosphatidyicholine is the susbtrate catalyzed by PLA2 to form LPC. Hence, reduced gastrointestinal concentrations of LPC can be realized by inhibiting phospholipase-A₂ in the gastrointestinal tract—preferably selectively or specifically, without inhibiting or essentially not inhibiting one or more other enzymes, such as PLD, PLC and PLB, that catalyze competing reactions. Preferably, in addition thereto, or independently thereof, the concentration and/or activity of one or more of the enzymes that catalyze competing reactions, such as PLD, PLC and PLB in various combinations, can be increased such that the amount of phosphatidylcholine substrate is reduced, and such that the concentration of LPC in the gastrointestinal lumen is likewise reduce.

As shown, lysophospholipases are enzymes that catabolize LPC directly. Hence, LPC can be directly modulated by enhancing the concentration and/or activity of the above-indicated lysophospholipases in the gastrointestinal lumen, so as to metabolize LPC to glycerophosphate and fatty acids, thereby lowering LPC concentration in the gastrointestinal lumen. Similarly, LPC can be directly modulated by enhancing the concentration and/or activity of the above-indicated lysophospholipases in plasma, such that LPC is catabolized to glycerophosphate and fatty acids, thereby lowering LPC concentration in plasma. Generally, the activity of lysophospholipase can be enhanced by adding exogenous lysophospholipases, e.g. by oral administration, and/or by other approaches known in the art.

Lysophospholipase enzyme are known: they can be of vegetal (Wang and Dennis 1999), bacterial or fungal (Witt, Mertsching et al. 1984; Witt, Schweingruber et al. 1984; Toyoda, Sugimoto et al. 1999; Flieger, Gong et al. 2001; Flieger, Neumeister et al. 2002), (Wright, Payne et al. 2004) (Duan and Borgstrom 1993; Ross and Kish 1994; Sunaga, Sugimoto et al. 1995; Dunlop, Muggli et al. 1997; Baker and Chang 1999; Taniyama, Shibata et al. 1999; Tosti, Dahl et al. 1999; Wang, Yang et al. 1999; Baker and Chang 2000; Gesta, Simon et al. 2002; Tokurnura, Kanaya et al. 2002; Tokumura, Majima et al. 2002; Shanado, Kometani et al. 2004; Sakagami, Aoki et al. 2005) or animal origin (Baker, 2002), (Baker, 1999), (Xie, 2004), (Duan, 1993), (Sunaga, 1995), (Ross, 1994), (Shanado, 2004), (Tosti, 1999), (Wang, 1999). Citations for these references are included in the bibliography below, immediately preceding the examples.

These lysophospholipases can be produced by known methods, i.e. extraction and purification from physiological fluids or plant extracts, or recombinant techniques: the daily dosage of lysophospholipases to provide a significant rate of hydrolysis of LPC in the gastrointestinal tract is based on the lysophospholipase specific activity, the amount of LPC formed upon digestion and the rate of absorption by the duodenal and jejunal mucosa, the two main loci of absorption. In one embodiment, the lysophospholipase is encapsulated in a pharmaceutically acceptable matrix to first protect the integrity of the enzyme from the hydrolytic attack of digestive protease such as trypsin and pepsin, and second to avoid or minimize any potential immunogenic effects induced by lysophospholipases of non-mammal origin. The matrix is preferably of polymeric nature, stable in the GI tract and non toxic. Modes of encapsulation are described in the litterature (el Soda, Pannell et al. 1989; Shah 2000; Muzykantov 2001; Walde and Ichikawa 2001; McMorn and Hutchings 2004; Millan, Marinero et al. 2004). The characteristics of the matrix are selected so as to allow the substrate and metabolites (e.g. LPC, glycerophophate-choline and fatty acids) to freely permeate within the capsule while retaining the immobilized lysophospholipase within the capsule porosity volume. Typically the enzymatic activity of the encapsulated lysophospholipase is measured in a simulated GI mimic using enzymology methods typical of what is described for the characterization of gastric and pancreatic lipases (Carriere 1993; Carriere, Renou et al. 2000). The oral dosage is such that the overall lysophospholipase activity is comprised between 100 to 100,000 micromoles of LPC hydrolyzed per minute).

Effective Amount of Lysophosphatidylcholine Modulation

The present invention relates to methods for treating a lysophosphatidylcholine-related condition, e.g., a diet-induced condition in which lysophosphatidylcholine plays a physiological role, by administering an effective amount of a lysophosphatidylcholine modulator to achieve therapeutic and/or prophylactic benefit in at least one condition being treated. Lysophosphatidylcholine modulators include agents that directly or indirectly act on lysophosphatidylcholine, phospholipase A2, or both. The actual amount effective for a particular application will depend on the condition being treated and the approach used. Determination of an effective amount is well within the capabilities of those skilled in the art, especially in light of the disclosure herein.

For example, a person of skill in the art can determine the amount of lysophosphatidylcholine modulation required to produce a therapeutic and/or prophylactic benefit in treating at least one of an insulin-related condition (e.g., diabetes and diabetes type 2) and/or a weight-related condition (e.g., unwanted weight gain and obesity). The amount of reduction, for example, can be determined by measuring a metabolite whose amount is affected by LPC modulation, e.g, the amount of lysophosphatidylcholine. The amount of LPC can be determined, for example, by measuring small intestine, lymphatic and/or serum levels post-prandially. Another technique for determining in vivo lysophosphatidylcholine levels involves taking direct fluid samples from the gastrointestinal tract. Other techniques would be apparent to one of skill in the art.

The effective amount for use in humans can be determined from animal models. For example, treatment of humans can be achieved by attaining circulating and/or gastrointestinal concentrations that have been found to be effective in animals and a dose for humans can be formulated accordingly.

The effective amount when referring to producing a benefit in treating an insulin-related condition (e.g., diabetes) and/or a weight-related condition (e.g., unwanted weight gain) will generally mean the levels that achieve clinical results recommended or approved (with respect to at least one condition being treated) by any of the various regulatory or advisory organizations in the medical or pharmaceutical arts (e.g., FDA, AMA) or by the manufacturer or supplier. For example, the effective amount when referring to a lysophosphatidylcholine modulator of the present invention will generally mean the dose ranges, modes of administration, formulations, etc., that have been recommended or approved by such organizations with respect to at least one condition being treated. Effective amounts of phospholipase inhibitors can be found, for example, in the Physicians Desk Reference.

A skilled person using techniques known in the art can determine the effective amount of the lysophosphatidylcholine modulator. For example, in some embodiments, the recommended dosage of an LPC modulator is between about 0.1 mg/kg/day and about 1,000 mg/kg/day. A person of skill in the art would be able to monitor in a patient the effect of a lysophosphatidylcholine modulator, as discussed above.

Pharmaceutical compositions suitable for use in the present invention include compositions wherein the active ingredients are present in an effective amount, i.e., in an amount effective to achieve therapeutic or prophylactic benefit in at least one condition being treated. The actual amount effective for a particular application will depend on the condition being treated and the route of administration.

The lysophosphatidylcholine modulators useful in the present invention, or pharmaceutically acceptable salts thereof, can be delivered to the patient using a number of routes or modes of administration. The term “pharmaceutically acceptable salt” means those salts which retain the biological effectiveness and properties of the compounds used in the present invention, and which are not biologically or otherwise undesirable. Such salts include salts with inorganic or organic acids, such as hydrochloric acid, hydrobromic acid, phosphoric acid, nitric acid, sulfuric acid, methanesulfonic acid, p-toluenesulfonic acid, acetic acid, fumaric acid, succinic acid, lactic acid, mandelic acid, malic acid, citric acid, tartaric acid or maleic acid. In addition, if the compounds used in the present invention contain a carboxy group or other acidic group, it may be converted into a pharmaceutically acceptable addition salt with inorganic or organic bases. Examples of suitable bases include sodium hydroxide, potassium hydroxide, ammonia, cyclohexylamine, dicyclohexyl-amine, ethanolamine, diethanolamine and triethanolamine.

If necessary or desirable, the lysophosphatidylcholine modulators may be administered in combination with other therapeutic agents. The choice of therapeutic agents that can be co-administered with the compositions of the invention will depend, in part, on the condition being treated.

The lysophosphatidylcholine modulators (or pharmaceutically acceptable salts thereof) may be administered per se or in the form of a pharmaceutical composition wherein the active compound(s) is in admixture or mixture with one or more pharmaceutically acceptable carriers, excipients or diluents. Pharmaceutical compositions for use in accordance with the present invention may be formulated in conventional manner using one or more physiologically acceptable carriers compromising excipients and auxiliaries, which facilitate processing of the active compounds into preparations which can be used pharmaceutically. Proper formulation is dependent upon the route of administration chosen.

For oral administration, the compounds can be formulated readily by combining the active compound(s) with pharmaceutically acceptable carriers well known in the art. Such carriers enable the compounds of the invention to be formulated as tablets, pills, dragees, capsules, liquids, gels, syrups, slurries, suspensions, wafers, and the like, for oral ingestion by a patient to be treated. In one embodiment, the oral formulation does not have an enteric coating. Pharmaceutical preparations for oral use can be obtained as a solid excipient, optionally grinding a resulting mixture, and processing the mixture of granules, after adding suitable auxiliaries, if desired, to obtain tablets or dragee cores. Suitable excipients are, in particular, fillers such as sugars, including lactose, sucrose, mannitol, or sorbitol; cellulose preparations such as, for example, maize starch, wheat starch, rice starch, potato starch, gelatin, gum tragacanth, mehtyl cellulose, hydroxypropylmethyl-cellulose, sodium carboxymethylcellulose, and/or polyvinyl pyrrolidone (PVP). If desired, disintegrating agents may be added, such as the cross-linked polyvinyl pyrrolidone, agar, or alginic acid or a salt thereof such as sodium alginate.

### BIBLIOGRAPHY

The following references describe knowledge known in the art that relates to the present invention, for example, as indicated above. These references are incorporated by reference herein.
Baker, R. R. and H. Chang (2000). “A metabolic path for the degradation of lysophosphatidic acid, an inhibitor of lysophosphatidylcholine lysophospholipase, in neuronal nuclei of cerebral cortex.” Biochim Biophys Acta 1483(1): 58-68.
Baker, R. R. and H. Y. Chang (1999). “Evidence for two distinct lysophospholipase activities that degrade lysophosphatidylcholine and lysophosphatidic acid in neuronal nuclei of cerebral cortex.” Biochim Biophys Acta 1438(2): 253-63.
Carriere (1993). “Secretion and contribution to Lipolysis of Gastic and Pancreatic Lipases During a Test Meal in Humans.” Gastroenterology: 876-888.
Carriere, F., C. Renou, et al. (2000). “The specific activities of human digestive lipases measured from the in vivo and in vitro lipolysis of test meals.” Gastroenterologv 119(4): 949-60.
Duan, R. D. and B. Borgstrom (1993). “Is there a specific lysophospholipase in human pancreatic juice?” Biochim Biophys Acta 1167(3): 326-30.
Dunlop, M. E., E. Muggli, et al. (1997). “Differential disposition of lysophosphatidylcholine in diabetes compared with raised glucose: implications for prostaglandin production in the diabetic kidney glomerulus in vivo.” Biochim Biophys Acta 1345(3): 306-16.
el Soda, M., L. Pannell, et al. (1989). “Microencapsulated enzyme systems for the acceleration of cheese ripening.” J Microencapsul 6(3): 319-26.
Flieger, A., S. Gong, et al. (2001). “Novel lysophospholipase A secreted by Legionella pneumophila.” J Bacteriol 183(6): 2121-4.
Flieger, A., B. Neumeister, et al. (2002). “Characterization of the gene encoding the major secreted lysophospholipase A of Legionella pneumophila and its role in detoxification of lysophosphatidylcholine.” Infect Immun 70(11): 6094-106.
Gesta, S., M. F. Simon, et al. (2002). “Secretion of a lysophospholipase D activity by adipocytes: involvement in lysophosphatidic acid synthesis.” J Lipid Res 43(6): 904-10.
McMom, P. and G. J. Hutchings (2004). “Heterogeneous enantioselective catalysts: strategies for the immobilisation of homogeneous catalysts.” Chem Soc Rev 33(2): 108-22.
Millan, C. G., M. L. Marinero, et al. (2004). “Drug, enzyme and peptide delivery using erythrocytes as carriers.” J Control Release 95(1): 27-49.
Muzykantov, V. R. (2001). “Delivery of antioxidant enzyme proteins to the lung.” Antioxid Redox Signal 3(1): 39-62.
Ross, B. M. and S. J. Kish (1994). “Characterization of lysophospholipid metabolizing enzymes in human brain.” J Neurochem 63(5): 1839-48.
Sakagami, H., J. Aoki, et al. (2005). “Biochemical and molecular characterization of a novel choline-specific glycerophosphodiester phosphodiesterase belonging to the nucleotide pyrophosphatase/phosphodiesterase (NPP) family.” J Biol. Chem.
Shah, N. P. (2000). “Probiotic bacteria: selective enumeration and survival in dairy foods.” J Dairy Sci 83(4): 894-907.
Shanado, Y., M. Kometani, et al. (2004). “Lysophospholipase I identified as a ghrelin deacylation enzyme in rat stomach.” Biochem Biophys Res Commun 325(4): 1487-94.
Sunaga, H., H. Sugimoto, et al. (1995). “Purification and properties of lysophospholipase isoenzymes from pig gastric mucosa.” Biochem J 308 (Pt 2): 551-7.
Taniyama, Y., S. Shibata, et al. (1999). “Cloning and expression of a novel lysophospholipase which structurally resembles lecithin cholesterol acyltransferase.” Biochem Biophys Res Commun 257(1): 50-6.
Tokumura, A., Y. Kanaya, et al. (2002). “Increased formation of lysophosphatidic acids by lysophospholipase D in serum of hypercholesterolemic rabbits.” J Lipid Res 43(2): 307-15.
Tokumura, A., E. Majima, et al. (2002). “Identification of human plasma lysophospholipase D, a lysophosphatidic acid-producing enzyme, as autotaxin, a multifunctional phosphodiesterase.” J Biol Chem 277(42): 39436-42.
Tosti, E., L. Dahl, et al. (1999). “Endothelial degradation of extracellular lysophosphatidylcholine.” Scand J Clin Lab Invest 59(4): 249-57.
Toyoda, T., H. Sugimoto, et al. (1999). “Sequence, expression in Escherichia coli, and characterization of lysophospholipase II.” Biochim Biophys Acta 1437(2): 182-93.
Walde, P. and S. Ichikawa (2001). “Enzymes inside lipid vesicles: preparation, reactivity and applications.” Biomol Eng 18(4): 143-77.
Wang, A. and E. A. Dennis (1999). “Mammalian lysophospholipases.” Biochim Biophys Acta 1439(1): 1-16.
Wang, A., H. C. Yang, et al. (1999). “A specific human lysophospholipase: cDNA cloning, tissue distribution and kinetic characterization.” Biochim Biophys Acta 1437(2): 157-69.
Witt, W., A. Mertsching, et al. (1984). “Secretion of phospholipase B from Saccharomyces cerevisiae.” Biochim Biophys Acta 795(1): 117-24.
Witt, W., M. E. Schweingruber, et al. (1984). “Phospholipase B from the plasma membrane of Saccharomyces cerevisiae. Separation of two forms with different carbohydrate content.” Biochim Biophys Acta 795(1): 108-16.
Wright, L. C., J. Payne, et al. (2004). “Cryptococcal phospholipases: a novel lysophospholipase discovered in the pathogenic fungus Cryptococcus gattii.” Biochem J 384(Pt 2): 377-84.

### EXAMPLES

The following examples are intended to illustrate details of the invention, without thereby limiting it in any manner.

### Example 1

### Absorption of Lysophosphatidylcholine was Reduced in Phospholipase A2-Deficient Mice

Phosphatidyl [³H]choline absorption as lysophosphatidyl [³H]choline occurred to a lesser extent in PLA2-deficient mice compared to wild-type (PLA2^{+/+}) mice as measured by LPC levels in portal vein blood and the liver. PLA2^{+/+} and PLA2^{-/-} mice were fed a lipid-rich meal containing 2.6 mM phosphatidyl [³H]choline, 2.6 mM cholesterol, and 13.33 mM triolein by stomach gavage. The animals were sacrificed after 2 hr, an aliquot of the portal vein blood and the liver were obtained for lipid extraction. The lipid extracts were separated by thin layer chromatography on silica gel G plates using methanol/ammonium/0.5% NaCl (50:5:50) as the solvent. The lipid spot co-migrating with standard lysophosphatidylcholine was scraped from the plates for scintillation counting to identify the presence of lysophosphatidyl[³H]choline. See FIG. 1.

### Example 2

### Insulin Sensitivity was Increased in Phospholipase A2-Deficient Mice

Insulin sensitivity was shown to increase in female PLA2-deficient mice compared with wild-type mice when fed a chow diet (FIG. 2(A)) or a Western-type high fat/high carbohydrate diet (FIG. 2(B)) for 14 weeks. Animals were injected ip with bovine insulin (1 U/kg body wt) after a 4-hour fast. Blood was obtained from tail veins for glucose analysis. Data were expressed as % fasting glucose levels (means±SD) from 8-10 animals in each group fed the chow diet and 5 animals in each group fed the high fat/high carbohydrate diet. See FIG. 2. * Significant difference from wild-type animals, P<0.05.

### Example 3

### Glucose Tolerance was Improved in Phospholipase A2-Deficient Mice

PLA2^{-/-} mice showed improved glucose tolerance compared with PLA2^{+/+} mice at 4-6 weeks on a Western-type diet (panel C), and at 16 weeks when on a Chow diet (panels B and D). Male PLA2^{+/+} wild type and PLA2^{-/-} knockout mice were maintained on low fat basal chow diet (panels A, B, and D) or high fat/high cholesterol Western-type diet (panel C) for at least 4 weeks. The animals were fasted overnight prior to experiments. Glucose tolerance tests were initiated by feeding each mouse with an oral dose of glucose (2 g/kg of body weight) in phosphate-buffered saline solution (panels A-C) or in a solution containing 2.6 mM phosphatidylcholine, 2.6 mM cholesterol, and 13.33 mM triolein (panel D). Blood was obtained from the tail vein before and at various time intervals for 2 hours after glucose administration. Glucose level was measured by enzymatic kit. See FIG. 3.

### Example 4

### Tissue Glucose Levels were Increased in Phospholipase A2-Deficient Mice

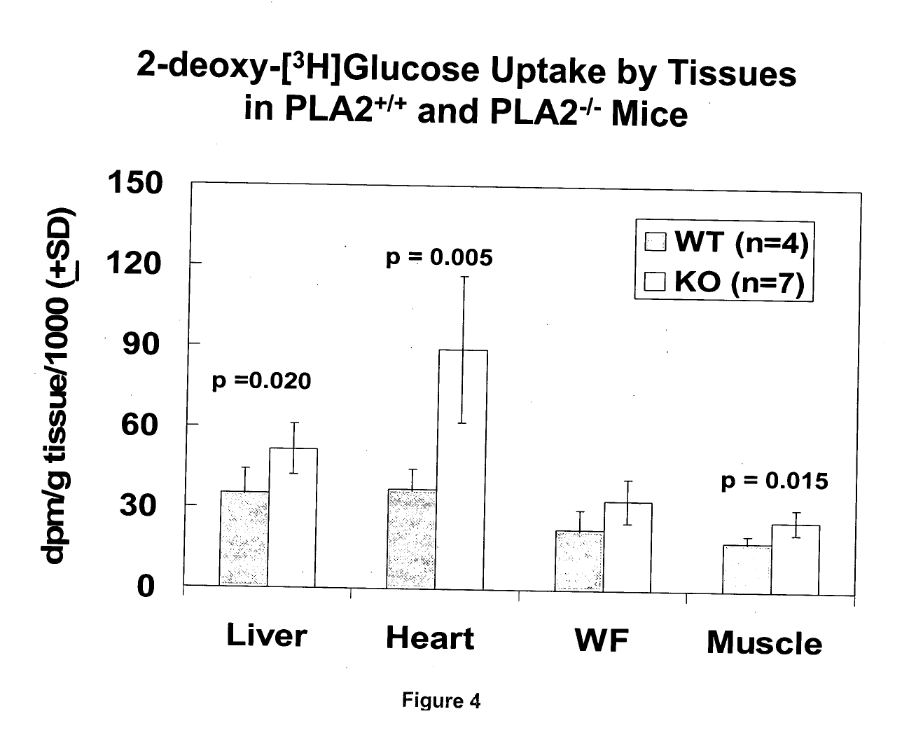

Glucose uptake by liver, heart, white fat and muscle each increased in PLA2^{-/-} mice compared to PLA2^{+/+} mice. In these experiments, 4-month old PLA2^{+/+} and PLA2^{-/-} mice fed the basal low fat/low cholesterol diet were injected intraperitoneally with a bolus load of glucose (2 g/kg body weight) containing 5 μCi 2-deoxy-[3H]glucose after an overnight fast. The animals were sacrificed after 30 min. Tissues were harvested and the amount of the radiolabeled glucose taken up by each tissue was quantitated by scintillation counting. See FIG. 4.

### Example 5

### Insulin-Stimulated Glucose Metabolism Increased Under Conditions of Reduced Levels of Lysophosphatidylcholine in (a) HepG2 Cells; (b) Myotube; and (c) 3T3L1 Adipocytes

Effects of LPC on glucose metabolism were assessed using (a) HepG2 cells as a model of liver cells, (b) differentiated L6 myotubes as a model of skeletal muscle cells and (c) differentiated 3T3L1 cells as a model of adipocytes. To assess usage of glucose for glycogen biosynthesis, cells were incubated in Krebs Ringer Hepes medium with 5 mM [¹⁴C]glucose, 100 nM insulin, and varying concentrations of LPC. At the end of the incubation period, the cells were washed and detached from the plates with 30% KOH. Carrier glycogen was then added to the cell lysate at a concentration of 40 mg/ml and then boiled for 30 min. Glycogen was precipitated from the solution by the addition of 800 μl 95% ice-cold ethanol and centrifuged at 1000×g. The pellet was re-suspended in buffer, and an aliquot of the sample was counted in a scintillation counter to determine the amount of [¹⁴C]glucose converted to glycogen. Glucose metabolism by differentiated 3T3L1 cells was assessed based on cellular uptake of [³H]deoxyglucose. The 3T3L1 cells were incubated in Krebs Ringer Hepes medium with 5 mM [³H]deoxyglucose, 100 nM insulin, and varying concentration of LPC. Glucose uptake was assessed based on cellular accumulation of [³H]deoxyglucose after a 30 min incubation period. See FIGS. 5(a), 5(b) and 5(c).

### Example 6

### Post-Prandial Fat Absorption was Reduced in Phospholipase A2-Deficient Mice on a High Fat Diet

Fat absorption was determined by measuring the appearance of [³H]triglyceride after an intragastric gavage of [3H]triglyceride in olive oil. After an overnight fast, male PLA2^{+/+} and PLA2^{-/-} mice on a chow (FIG. 6(A)) or Western-type high fat (FIG. 6(B)) diet for 4 weeks were injected via the retroorbital plexus with 12.5 mg of Triton WR-1339. Ten minutes after injection, the mice received an intragastric load of 2 μCi of [³H]triolein in 50 μl of olive oil. Blood samples were taken 1, 2, 4, and 6 hours after gavage by tail bleeding. Radioactivity appearing in plasma was determined by liquid scintillation counting. Data are expressed as means±SD from 7 animals in each group fed the chow diet and 4 animals in each group fed the Western-type diet. See FIG. 6. * Significant difference from wild-type animals, P<0.05.

### Example 7

### Weight Gain in Phospholipase A2-Deficient Mice on a High Fat Diet was Lower than that of (a) Wild-Type (+/+) and (b) Heterozygous (+/−) Mice

(a) Male (FIG. 7(a)(A)) and female (FIG. 7(a)(B)) mice 8-10 weeks of age were placed on diet containing 21% fat and 0.15% cholesterol (wt/wt) for 16 weeks. Body weights of wild-type and PLA2-deificent mice were determined every 2 weeks. The inset in each panel shows weight gain in wild-type and PLA2-deficient mice after 16 weeks. Data points represent means±SD from 4 animals in each group. * Significant difference between the groups, P<0.05. See FIG. 7(a).

(b) Weight gain in PLA2-deficient female mice (PLA^{-/-}) on a Western diet decreased compared to wild type PLA2^{+/+} or heterozygous (PLA2^{+/-}) female mice on such a diet. Female PLA2^{+/+} wild type (WT), heterozygous PLA2^{+/-} (HET), and homozygous PLA2^{-/-} (KO) mice were placed on a Western type high fat/high cholesterol diet containing 21% fat and 0.15% cholesterol (wt/wt) at 6 weeks of age. Body weights were determined after 6 months on the diet. Data points represent mean±S.d. from 6 mice from each group. See FIG. 7(b).

### Example 8

### Weight Gain in Certain Tissues of Phospholipase A2-Deficient Mice on a High Fat Diet

Weight gain in various tissues of PLA2^{+/+} and PLA2^{-/-} mice on a high fat diet indicated that weight gain in white fat is significantly reduced in PLA2-deficinet mice. Wild type PLA2^{+/+} (WT) and homozygous PLA2^{-/-} mice (KO) were maintained on Western-type high fat/high cholesterol diet for 16 weeks. Tissues were removed and wet weights were recorded. Data are expressed as mean±S.D. from 4 animals in each group. * indicates significant difference at P<0.05. See FIG. 8.

### Example 9

### Insulin and Leptin Levels Decreased in Phospholipase A2-Deficient Mice Fed a Western Diet

Plasma insulin and leptin levels decreased in PLA2^{-/-} mice fed a Western diet compared to PLA2^{+/+} mice on such a diet. Animals were maintained on the high fat/high cholesterol Western-type diet for 16 weeks. On the day of the determination, animals were fasted for 4 to 6 hours prior to blood drawing to obtain plasma samples. Plasma leptin and insulin concentrations were measured using radioimmunassay kits from Linco Research (St. Charles, Mo.). Data was expressed as mean±S.D. from 10 mice in each group. See FIG. 9.

### Example 10

### Body Temperature and Food Intake Did not Significantly Change in Phospholipase A2-Deficient Mice

No significant differences were observed in the amount of food consumed per day or the resting body temperature between wild-type and PLA2-deficient mice fed a Western-type diet. Mice 8-10 weeks of age were placed on diet containing 21% fat and 0.15% cholesterol (wt/wt) for 16 weeks. Values represent means±SD from animals in each group. See FIG. 10.

### Example 11

### Phospholipid Absorption was not Lowered in Phospholipase A2-Deficient Mice

Phospholipid absorption from intestinal lumen to lymphatics in PLA2-deficient mice was compared with that in wild-type mice. Lymph fistula of wild-type and PLA2-deficient mice were infused with a test meal containing 35 μg/mL of palmitoyl-2-[¹⁴C]oleoyl-phosphatidylcholine and 5 μg/mL of cholesterol. Lymph was collected for 1 hour before lipid infusion and served as the fasting lymph. After the onset of lipid infusion, lymph was collected every 2 hours during the first 4 hours and then hourly for the remaining time period. Aliquots of the lymph were taken for radioactivity determination by scintillation counting to determine the amount of radiolabel absorbed. Data points represent means with standard deviation form 4 wild-type and three PLA2^{-/-} mice. See FIG. 11.

### Example 12

### Cholesterol Absorption Decreased Using (a) the Phospholipase Inhibitor, FPL 67047XX, But (b) not in Phospholipase A2-Deficient Mice, when Evaluated in Mice on a High-Fat Diet

Cholesterol transport from intestinal lumen to lymphatics in rats was decreased using FPL 6704XX. Lymph fistula rats were infused with an emulsion that consisted of 40 μmol triolein, 7.8 μmol of egg phosphatidylcholine, and 7.8 μmol [¹⁴C] cholesterol in 3.0 mL of phosphate-buffered saline in the absence or presence of the PLA2 inhibitor FPL 67047XX. Lymph was collected for 1 hour before lipid infusion and served as the fasting lymph. After the onset of lipid infusion, lymph was collected every 2 hours during the first 4 hours and then hourly for the remaining time period. Aliquots of the lymph were taken for radioactivity determination by scintillation counting to determine the amount of radio-label absorbed. Data points represent means±SD. See FIG. 12(a).

Nonetheless, cholesterol plasma levels did not decrease in PLA2-deficient mice fed a Western diet. Six male wild type (WT) and six male PLA2 knockout mice (KO) at 6 weeks of age were placed on a Western-type high fat/high cholesterol diet containing 21% fat and 0.15% cholesterol. Plasma was obtained from the animals prior to the initiation of the diet to obtain baseline values, and at 5 and 11 months after placement of the test diet. On the day of the determinations, animals were fasted for 4 to 6 hours before blood was drawn to obtain plasma samples. Plasma cholesterol values were determined by colorimetric assays with kits from Wako Chemicals (Richmond, Va.). See FIG. 12(b).

All publications, patents, and patent applications mentioned in this specification are herein incorporated by reference to the same extent as if each individual publication, patent or patent application was specifically and individually indicated as being incorporated by reference.

It will be apparent to one of ordinary skill in the art that many changes and modifications can be made thereto without departing from the spirit or scope of the appended claims, and such changes and modifications are contemplated as being contained within the scope of this invention.

1. A method of modulating lysophosphatidylcholine in a subject, the method comprising:
(a) modulating plasma lysophosphatidylcholine concentration or activity; or
(b) reducing gastrointestinal lysophosphatidylcholine concentration by inhibiting phospholipase-A₂ in the gastrointestinal tract, without inhibiting or essentially not inhibiting a gastrointestinal non-PLA₂ phospholipase having activity for hydrolysis of phosphatidylcholine to products other than lysophosphatidylcholine; or
(c) reducing gastrointestinal lysophosphatidylcholine concentration by inhibiting phospholipase-A₂ in the gastrointestinal tract, without inhibiting or essentially not inhibiting a gastrointestinal lipase having activity for catabolizing phosphatidylcholine to products other than lysophosphatidylcholine; or
(d) reducing gastrointestinal lysophosphatidylcholine concentration by increasing the concentration or activity of a gastrointestinal non-PLA₂ phospholipase having activity for hydrolysis of phosphatidylcholine to products other than lysophosphatidylcholine; or
(e) modulating the concentration or activity of lysophosphatidylcholine by administering a lysophosphatidylcholine modulating agent that acts directly on lysophosphatidylcholine; or
(f) combinations thereof.

2. The method of claim 1 wherein the lysophosphatidylcholine is modulated in the subject by reducing lysophosphatidylcholine absorption in the gastrointestinal tract.

3. The method of claim 1 wherein the lysophosphatidylcholine is modulated in the subject by reducing the effectiveness of lysophosphatidylcholine as a signaling messenger.

4. The method of claim 1 wherein the lysophosphatidylcholine is modulated in the subject by reducing the activity of lysophosphatidylcholine in signaling pathways.

5. The method of claim 1 wherein lysophosphatidylcholine is modulated by reducing lysophosphatidylcholine concentration or activity without affecting or without substantially affecting one or both of cholesterol absorption or cholesterol absorption efficiency.

6. The method of claim 1 wherein lysophosphatidylcholine is modulated by reducing lysophosphatidylcholine concentration or activity without significantly lowering one or both of phospholipid absorption or phospholipids absorption efficiency.

7. The method of claim 1 wherein lysophosphatidylcholine is modulated by a method that includes inhibiting phospholipase-A₂ in the gastrointestinal tract, without effect or with essentially no effect on one or more of fat absorption or on the absorption of fat-soluble vitamins.

8. The method of claim 1 wherein lysophosphatidylcholine is modulated by a method that includes inhibiting phospholipase-A₂ in the gastrointestinal tract, without inhibiting or essentially not inhibiting gastrointestinal phospholipase B.

9. The method of claim 1 wherein lysophosphatidylcholine is modulated by a method that includes inhibiting phospholipase-A₂ in the gastrointestinal tract, without inhibiting or essentially not inhibiting gastrointestinal phospholipase-A₁.

10. The method of claim 1 wherein lysophosphatidylcholine is modulated by a method that includes inhibiting phospholipase-A₂ in the gastrointestinal tract, without inhibiting or essentially not inhibiting any other gastrointestinal phospholipases.

11. The method of claim 1 wherein lysophosphatidylcholine is modulated by a method that includes inhibiting phospholipase-A₂ in the gastrointestinal tract, without inhibiting or essentially not inhibiting gastrointestinal carboxyl ester lipase.

12. The method of claim 1 wherein lysophosphatidylcholine is modulated by a method that includes inhibiting phospholipase-A₂ in the gastrointestinal tract, without inhibiting or essentially not inhibiting gastrointestinal pancreatic triglyceride lipase.

13. The method of claim 1 wherein lysophosphatidylcholine is modulated by a method that includes inhibiting phospholipase-A₂ in the gastrointestinal tract, without inhibiting or essentially not inhibiting any other gastrointestinal lipases.

14. The method of claim 1 comprising increasing the concentration or activity of gastrointestinal phospholipase B.

15. The method of claim 1 comprising increasing the concentration or activity of gastrointestinal phospholipase A1.

16. The method of claim 1 wherein lysophosphatidylcholine is modulated using antibodies.

17. The method of claim 1 wherein both the concentration and activity of lysophosphatidylcholine are reduced.

18. A method of treating a condition comprising modulating lysophosphatidylcholine activity or concentration in a subject by
(a) modulating plasma lysophosphatidylcholine concentration or activity; or
(b) reducing gastrointestinal lysophosphatidylcholine concentration by inhibiting phospholipase-A₂ in the gastrointestinal tract, without inhibiting or essentially not inhibiting a gastrointestinal non-PLA₂ phospholipase having activity for hydrolysis of phosphatidylcholine to products other than lysophosphatidylcholine; or
(c) reducing gastrointestinal lysophosphatidylcholine concentration by inhibiting phospholipase-A₂ in the gastrointestinal tract, without inhibiting or essentially not inhibiting a gastrointestinal lipase having activity for catabolizing phosphatidylcholine to products other than lysophosphatidylcholine; or
(d) reducing gastrointestinal lysophosphatidylcholine concentration by increasing the concentration or activity of a gastrointestinal non-PLA₂ phospholipase having activity for catabolizing phosphatidylcholine to products other than lysophosphatidylcholine; or
(e) modulating the concentration or activity of lysophosphatidylcholine by administering a lysophosphatidylcholine modulating agent that acts directly on lysophosphatidylcholine; or
(f) combinations thereof.

19. The method as recited in claim 18 wherein said condition is a diet-related condition.

20. The method as recited in claim 18 wherein said condition is an insulin-related condition.

21. The method as recited in claim 18 wherein said condition is diabetes.

22. The method as recited in claim 18 wherein said condition is diabetes type 2.

23. The method as recited in claim 18 wherein said condition is a weight-related condition.

24. The method as recited in claim 18 wherein said condition is obesity.

25. The method as recited in claim 18 wherein said condition is weight gain.

26. The method as recited in claim 18 wherein said condition is hyperlipidemia.

27. The method as recited in claim 18 wherein the concentration or activity of lysophosphatidylcholine is reduced by the method of any of claims 2 through 17.

28. The method as recited in claim 18 wherein said reducing lysophosphatidylcholine results in increased insulin sensitivity in said subject.

29. The method as recited in claim 18 wherein said reducing lysophosphatidylcholine results in decreased post-prandial blood glucose levels in said subject.

30. The method as recited in claim 18 wherein said reducing lysophosphatidylcholine results in improved glucose tolerance in said subject.

31. The method as recited in claim 18 wherein said reducing lysophosphatidylcholine results in decreased fasting blood glucose levels in said subject.

32. The method as recited in claim 18 wherein said reducing lysophosphatidylcholine results in increased insulin-stimulated glucose metabolism in said subject.

33. The method as recited in claim 18 wherein said reducing lysophosphatidylcholine results in increased insulin-stimulated glucose metabolism in adipocytes of said subject.

34. The method as recited in claim 18 wherein said reducing lysophosphatidylcholine results in increased tissue glucose metabolism in said subject.

35. The method as recited in claim 18 wherein said reducing lysophosphatidylcholine results in decreased fasting blood insulin levels in said subject.

36. The method as recited in claim 18 wherein said subject is insulin resistant and said reducing lysophosphatidylcholine results in decreased fasting blood insulin levels in said subject.

37. The method as recited in claim 18 wherein said reducing lysophosphatidylcholine results in decreased fat absorption in said subject when on a high fat diet.

38. The method as recited in claim 18 wherein said reducing lysophosphatidylcholine results in a decrease in weight gain in said subject when on a high fat diet.

39. The method as recited in claim 38 wherein said decrease in weight gain occurs without a significant change in food intake of said subject.

40. The method as recited in claim 38 wherein said decrease in weight gain occurs without a significant change in energy expenditure of said subject.

41. The method as recited in claim 18 wherein said reducing lysophosphatidylcholine results in a decrease in weight gain in white fat of said subject when on a high fat diet.

42. The method as recited in claim 18 wherein said reducing lysophosphatidylcholine results in a decrease in leptin levels.

43. The method as recited in claim 18 wherein said reducing lysophosphatidylcholine results in a decrease in leptin levels when on a high fat diet.

44. The method as recited in claim 18 wherein said reducing lysophosphatidylcholine does not result in a significant change in body temperature of said subject.

45. The method as recited in claim 18 wherein said reducing lysophosphatidylcholine does not result in a significant lowering of phospholipid absorption of said subject.

46. The method as recited in claim 18 wherein said reducing lysophosphatidylcholine does not result in a significant decrease in cholesterol absorption of said subject.

47. The method as recited in claim 18 wherein said reducing lysophosphatidylcholine does not result in a significant decrease in cholesterol absorption of said subject when on a high fat diet.
